(19) United States
(12) Patent Application Publication
(10) Pub. No.: US 2024/0424056 A1
(43) Pub. Date: Dec. 26, 2024

(54) Peptides for Anti Fibrotic Therapy

(71) Applicant: McMaster University, Hamilton (CA)

(72) Inventors: Joan KREPINSKY, Hamilton (CA); Jackie TRINK, Hamilton (CA)

(21) Appl. No.: 18/291,069

(22) PCT Filed: Jul. 22, 2022
(86) PCT No.: PCT/CA2022/051139

§ 371 Date: Jan. 22, 2024

Related U.S. Application Data
(60) Provisional application No. 63/203,462, filed on Jul. 23, 2021.

(51) Int. Cl. A61K 38/17; A61P 1/16; A61P 11/00; C07K 16/18; C12N 15/113

(52) U.S. Cl. CPC A61K 38/177; A61P 1/16; A61P 11/00; C07K 16/18; C12N 15/113

(57) ABSTRACT

A method of treating or inhibiting the development of fibrosis in a subject is provided. The method comprises administering to the subject a therapeutically effective amount of an entity that disrupts the interaction between activated alpha 2-macroglobulin (α2M*) and cell surface 78 kDa glucose regulated protein csGRP78. In one embodiment, administration of novel peptides that block activated α2-macroglobulin (α2M*) interaction with csGRP78 are provided.

### FIELD OF INVENTION

The present invention generally relates to anti-fibrotic therapy, and more particularly relates to peptides which are useful to treat and/or prevent fibrosis.

### BACKGROUND OF THE INVENTION

Diabetic kidney disease (DKD) is the leading cause of kidney failure in developed nations, with patients suffering the highest morbidity and mortality rates of any kidney failure patient group. Currently, treatment can only delay DKD progression. Thus, there is a major need to identify new therapeutic targets for this disease. The earliest pathologic hallmarks of DKD include glomerular hypertrophy and basement membrane thickening, followed by glomerular sclerosis due to deposition of extracellular matrix (ECM) proteins. Glomerular mesangial cells (MC) play a central role in the pathogenesis of glomerular sclerosis in DKD. While insight into the molecular mechanisms involved in MC matrix synthesis in response to high glucose (HG) has been gained, the identification of clinically translatable targets is still much needed.

Current standard of care for DKD patients includes treatment of hyperglycemia to levels as near normal as possible. However, maintaining normal blood glucose levels is often difficult to both achieve and sustain, and targeting normal glucose levels is associated with the complication of low blood glucose, or hypoglycemia. The administration of ACE inhibitors and ARBs are used to delay the progression of DKD, although their use is somewhat limited by complications (hyperkalemia and worsening kidney function). They can also be considered nonspecific treatments as they additionally regulate blood pressure. Recently, the use of SGLT2 inhibitors has been accepted as a prescribed treatment for DKD patients, but is limited to those with type II diabetes. Adverse effects, including urogenital infections, have also somewhat limited their use. Even with this current standard of care, many DKD patients still progress towards and reach End Stage Kidney Disease limiting the success to prevent disease onset or progression of current treatment options. Thus, new therapeutic interventions must be identified.

The endoplasmic reticulum (ER) chaperone 78 kDa glucose regulated protein (GRP78) maintains proper protein folding and homeostasis within the cell. It is now recognized that in non-homeostatic conditions, such as with ER stress, GRP78 can also translocate to the cell surface to act as a receptor for intracellular signaling. While best studied in tumor cells, it has recently been shown that cell surface (cs)GRP78 plays a role in HG-induced profibrotic responses by MC through activation of PI3K/Akt signaling. How HG induces intracellular signaling through csGRP78, however, has yet to be elucidated.

α2-macroglobulin (α2M) is an abundant serum protein that can bind to and inhibit a wide range of proteinases. At a molecular weight of 720 kDa, it is comprised of four identical 180 kDa subunits. Each subunit contains a bait region that is cleaved once bound by a proteinase. Upon cleavage of all 4 subunits, a conformational change occurs that entraps the proteinase. The resulting complex is considered the activated form of α2M, designated α2M*, in which receptor recognition sites are exposed that allow interaction with its two identified receptors, low density lipoprotein receptor-like protein (LRP1) and csGRP78. The binding affinity for csGRP78 is significantly higher at a Kd ˜100 pM compared with a Kd in the nM range for LRP1, the predominant role of which is the endocytic clearance of α2M *.

α2M* interaction with csGRP78 has been implicated predominantly in the pathogenesis of various cancers. α2M* binds to a region in the NH2-terminal domain of csGRP78 to initiate signaling pathways that promote tumor cell proliferation and survival such as ERK1/2, p38 MAPK, PI3K/Akt, and NF-κB. HG-induced PI3K/Akt activation and downstream matrix production in MC has been shown to require csGRP78, but the ligand that activates csGRP78 has yet to be identified.

It would be desirable to develop an effective anti-fibrotic therapy that would be useful to treat diabetic kidney as well as fibrotic disease in other organs.

### SUMMARY OF THE INVENTION

It has now been determined that disruption of the interaction between α2M* and csGRP78 is effective to inhibit the development of fibrosis in a subject.

Thus, in one aspect of the invention, a method of treating or inhibiting the development of fibrosis in a subject is provided, comprising administering to the subject a therapeutically effective amount of an entity that disrupts the interaction between α2M* and csGRP78.

In another aspect of the invention, an anti-fibrotic composition is provided comprising a peptide derived from the α2M* binding site in csGRP78 comprising an N-terminal cysteine, and a pharmaceutically acceptable carrier.

In a further aspect, a novel anti-fibrotic peptide is provided derived from the csGRP78 peptide, LIGRTWNDPSVQQDIKFL, wherein the anti-fibrotic peptide i) comprises an N-terminal cysteine residue, ii) comprises at least 10 consecutive amino acid residues of the csGRP78 peptide, iii) retains the tertiary structure of the csGRP78 peptide, and iv) comprises at least one amino acid substitution, deletion or addition to the csGRP78 peptide.

### BRIEF DESCRIPTION OF THE DRAWINGS

Embodiments of the invention are shown in more depth by the following descriptions to their respective drawings listed below.

FIG. 1 shows α2M is increased by HG in mesangial cells (MC) and in diabetic kidneys. HG increased α2M mRNA (24 h) (A) and protein (48 h) (B) expression in MC. No effect was seen with the osmotic control mannitol (M) (A: n=11; B: n=9) (*p<0.01 vs others). (C) α2M expression increased dose-dependently with HG (24 h) concentrations from 10 to 30 mM (n=3, *p<0.01 vs con). (D) α2M transcript expression, assessed by ISH, was significantly higher in type 1 diabetic Akita kidney sections compared to wild type (WT) mice at 40 weeks of age (40× magnification, n=3) (*p<0.01 vs control). (E) α2M protein expression, as assessed by IHC, was significantly higher in kidney sections of 30- and 40-week old type 1 diabetic Akita mice compared to wild type mice (40× magnification, n=10). (F) This was also seen by immunoblotting of kidney lysate from 40-week old Akita compared to wild type mice (n=3) (*p<0.01 or ** p<0.05 vs respective control).

FIG. 2 shows Activated α2M (α2M*) is increased by HG in mesangial cells and in diabetic kidney mesangium. (A) High glucose (HG, 48 h) increased media expression of α2M (left) and α2M* (right) in MC, seen using a nondenaturing gel. Activated α2M migrates faster on a non-denaturing gel compared to its inactive form. The osmotic control mannitol (M) had no effect (n=3) (** p<0.05 vs its own control). (B) α2M* expression was significantly higher in kidney sections from type 1 diabetic Akita mice compared to wild type (WT) mice at 30 and 40 weeks of age (40× magnification, n=10 each, ** p<0.05 vs respective control). (C) α2M* expression was also significantly higher in uninephrectomized type 1 diabetic CDI mice compared to their non-diabetic controls (40× magnification, n=3, *p<0.01 vs control group). (D) α2M* colocalization with mesangial cells, identified by their marker α8-integrin, was significantly higher in kidney sections from type 1 diabetic Akita mice compared to wild type mice at 40 weeks of age (40× magnification, n=2, 20 glomeruli per section). DAPI is the nuclear marker (** p<0.05 vs respective control). (E) α2M* expression in human biopsy samples from DKD patients was significantly higher compared to control kidneys (40× magnification, n=4) (** p<0.05 vs control group).

FIG. 3 shows that α2M knockdown or neutralization inhibits HG-induced Akt activation and downstream ECM accumulation. (A,B) Immunoblotting shows that α2M siRNA inhibited upregulation of fibronectin (FN), collagen (Col) IV, the cytokine connective tissue growth factor (CTGF) and Akt activation (pAkt on S473) compared to control siRNA in response to high glucose (HG, 48 h, n=5) (*p<0.01 vs all others in the individual group). (C) Fura 2-AM calcium assay in 1-LN cells shows that the increased release of intracellular calcium stores in response to α2M* (100 pM, 15 min) is inhibited with addition of the neutralizing antibody Fα2M (2 μg/ml), but not control IgG. Groups were compared at one minute after time of treatment which is indicated by the arrow (n=6, p *<0.01 or ** p<0.05). (D,E) Antibody neutralization of α2M* abrogated high glucose (HG, 48 h)-induced FN, Col IV and CTGF protein upregulation and Akt activation (pAkt on S473), compared with nonspecific IgG (n=11) (*p<0.01 or ** p<0.05 vs con and HG+Fα2M).

FIG. 4 shows that an α2M* blocking peptide inhibits HG-induced profibrotic responses. (A) The peptide (Pep) blocking csGRP78/α2M* interaction, but not a control scrambled (Scr) peptide (both 100 nM), inhibited release of intracellular calcium stores in response to α2M* (100 pM, 15 min) in 1-LN cells (n=6, *p<0.01 or ** p<0.05). (B,C) MC were treated with high glucose (HG, 48 h) with or without peptides as in (A). The inhibitory peptide, but not the scrambled peptide, prevented HG-induced Akt activation (B) and (C) upregulation of matrix proteins fibronectin (FN) and collagen (Col) IV and of the profibrotic cytokine connective tissue growth factor (CTGF) (n=5, *p<0.01 vs control and HG with peptide).

FIG. 5 shows LRP1 knockdown did not affect Akt activation or ECM production. (A,B) Knockdown of LRP1 with siRNA did not attenuate Akt activation (pAkt on S473) or production of fibronectin (FN), Collagen (Col) IV or connective tissue growth factor (CTGF) compared to control siRNA in response to high glucose (HG, 48 h). Successful LRP1 knockdown is shown (n=5, *p<0.01 or ** p<0.05 vs. all others in individual group).

FIG. 6 shows that increased matrix synthesis with csGRP78 overexpression requires α2M *. (A) α2M* (100 pM) induced cell surface expression of GRP78 at 3 and 6 h in MC (n=3, *p<0.01) (B) HG-induced Akt activation (pAkt on S473) was not augmented by addition of α2M* (100 pM, 24 h) (n=6, ** p<0.05 or *p<0.01 vs control). (C) Increased translocation of GRP78 to the cell surface was induced by overexpression of GRP78 lacking KDEL (ΔKDEL) in MC (D) This increased both basal and high glucose (HG, 48 h)-induced Akt activation (pAkt on S473). (E) Similar findings were seen with HG (48 h)-induced fibronectin (FN), collagen (Col) IV and connective tissue growth factor (CTGF) upregulation (for C, D, E: n=3, ** p<0.05 vs empty vector (EV) control (significant through t test) or *p<0.01 vs ΔKDEL vector control). (F) The augmented responses to HG seen with GRP78 ΔKDEL overexpression, including Akt activation (pAkt on S473) and collagen (Col) IV upregulation, were prevented by the α2M*/csGRP78 inhibitory peptide (Pep), but not the scrambled (Scr) control peptide (n=5, ** p<0.05 or *p<0.01). (G) Similar inhibition was seen with α2M* neutralization using Fα2M (2 μg/ml) (n=4, ** p<0.05 or *p<0.01).

FIG. 7 shows TGFβ1 production is dependent on α2M* in MC, with urinary TGFβ1/α2M* association in patients with established DKD. (A) Neutralization of α2M* with Fα2M (2 μg/ml) prevented high glucose (HG, 48 h)-induced secretion of TGFβ1 into the medium, as assessed by ELISA (n=4, **** p<0.0001). Control IgG had no effect. (B) There was an association between urinary α2M* and TGFβ1 in patients with established DKD (p=0.03, Pearson correlation).

FIG. 8 shows HG-induced csGRP78 expression on (A) proximal tubular cells and (B) renal fibroblasts with both cell types promoting tubulointerstitial fibrosis in late DKD, (C) attenuation of HG-induced FAK activation (phosphorylation of Tyr397) and ECM production by csGRP78 inhibition in tubular cells, (D) csGRP78 inhibition in renal fibroblasts prevented HG-induced FAK activation and matrix production, and that inhibitory peptide preventing interaction of csGRP78 and α2M* attenuated HG-induced Akt activation (phosphorylation at Ser473) and ECM production in both (E) proximal tubular cells and (F) renal fibroblasts.

FIG. 9 shows that α2M* is increased in ⅚ nephrectomized (Nx) mice, a model of non-diabetic chronic kidney disease (CKD). CD-1 mice underwent uninephrectomy followed by nephrectomy of ⅔ of the remaining kidney. Kidney α2M* was assessed after 10 weeks by IHC.

FIG. 10 shows that α2M* and α2M are increased in lung fibrosis by α2M* staining in the bleomycin model of lung fibrosis at 21 days, when fibrosis is readily apparent.

FIG. 11 illustrates: A) the results of a calcium signaling assay in 1LN cells which shows that the peptide without an N-terminal cysteine residue in Leu⁹⁸-Leu¹¹⁵ (Pep woC) did not inhibit calcium signaling, B) results showing that Pep woC does not prevent HG-induced profibrotic responses in MC, C) the results of calcium signaling which show that methionine in place of the N-terminal cysteine (M Pep), a peptide dimer (Dimer Pep) and a cyclic peptide (Cyclic Pep) do not inhibit calcium signaling, and D) results showing that these peptides also do not prevent HG-induced profibrotic responses in MC.

FIG. 12 is a schematic illustrating the proposed pathway for HG-induced α2M*/csGRP78 mediated profibrotic signaling in MC. HG leads to increased synthesis and activation of α2M, as well as translocation to the cell surface of its receptor GRP78. Interaction between α2M*/csGRP78 leads to activation of Akt and downstream synthesis of profibrotic cytokines and extracellular matrix proteins.

FIG. 13 provides the amino acid (A) and nucleic acid-encoding (B) sequences of human α2M; and

FIG. 14 provides the amino acid (A) and nucleic acid-encoding (B) sequences of human csGRP78-encoding nucleic acid sequences.

### DETAILED DESCRIPTION

A method of treating or inhibiting the development of fibrosis in a subject is provided, comprising administering to the subject a therapeutically effective amount of an entity that disrupts the interaction between activated α2-macroglobulin (α2M*) and cell surface 78 kDa glucose regulated protein (csGRP78).

The present method is applicable to treat or prevent fibrosis. As used herein, the term fibrosis, also known as fibrotic scarring, is meant to encompass pathological conditions in which connective tissue replaces normal parenymal tissue, leading to the excessive accumulation of extracellular matrix and, ultimately, the formation of permanent fibrotic scar. Fibrosis can occur in various organs, such as lung (pulmonary fibrosis such as cystic fibrosis or idiopathic pulmonary fibrosis), liver (such as fibrosis from steatohepatitis or autoimmune diseases), kidney (chronic kidney disease of any cause including diabetic kidney disease), heart (myocardial fibrosis such as following infarction), as well as in skin (such as scleroderma, keloid or nephrogenic systemic fibrosis) and bone marrow (myelofibrosis).

Alpha 2-macroglobulin (alpha 2M or α2M) is an abundant serum protein that can bind to and inhibit a wide range of proteinases. The term “alpha 2M” encompasses mammalian α2M, including human α2M and α2M of other mammalian species, as well as functionally equivalent variants and isoforms thereof. The activated form of alpha 2-macroglobulin (alpha 2M* or α2M*) comprises 4 subunits of alpha-2M. Human α2M has the amino acid sequence of NCBI Reference Sequence: NP_000005.3 (isoform a) encoded by at least gene sequence, NCBI Reference, NM_000014.6 (Variant 1) and gene sequence, NCBI Reference, NM_001347423 (Variant 2). Isoform b has the amino acid sequence of NCBI Reference, NP 001334353, and isoform c has the amino acid sequence of NCBI Reference NP 001334354.

GRP78 (78 kDa glucose regulated protein), also known as binding immunoglobulin protein (BiP) or heat shock 70 kDa protein 5 (HSPA5), is a molecular chaperone located in the endoplasmic reticulum. The term GRP78, or csGRP78, encompasses mammalian GRP78, including GRP78 of humans and other mammalian species, as well as functionally equivalent variants and isoforms thereof. Human GRP78 has the amino acid sequence of NCBI Reference Sequence: NP_005338 which is encoded by at least the gene sequence, NCBI Reference, NM_005347.

The present method includes the step of administering to a subject an entity that disrupts the interaction between α2M* and csGRP78. The entity may be any entity which disrupts, prevents or minimizes the interaction between α2M* and csGRP78, and which is physiologically acceptable. The entity may be a protein, including an antibody against either of α2M and csGRP78, a peptide such as a peptide that blocks the binding site of either of α2M and csGRP78 (and therefore the binding of α2M and csGRP78), a nucleic acid which prevents expression of α2M or of csGRP78, a small molecule that prevents the interaction of α2M and csGRP78, and the like.

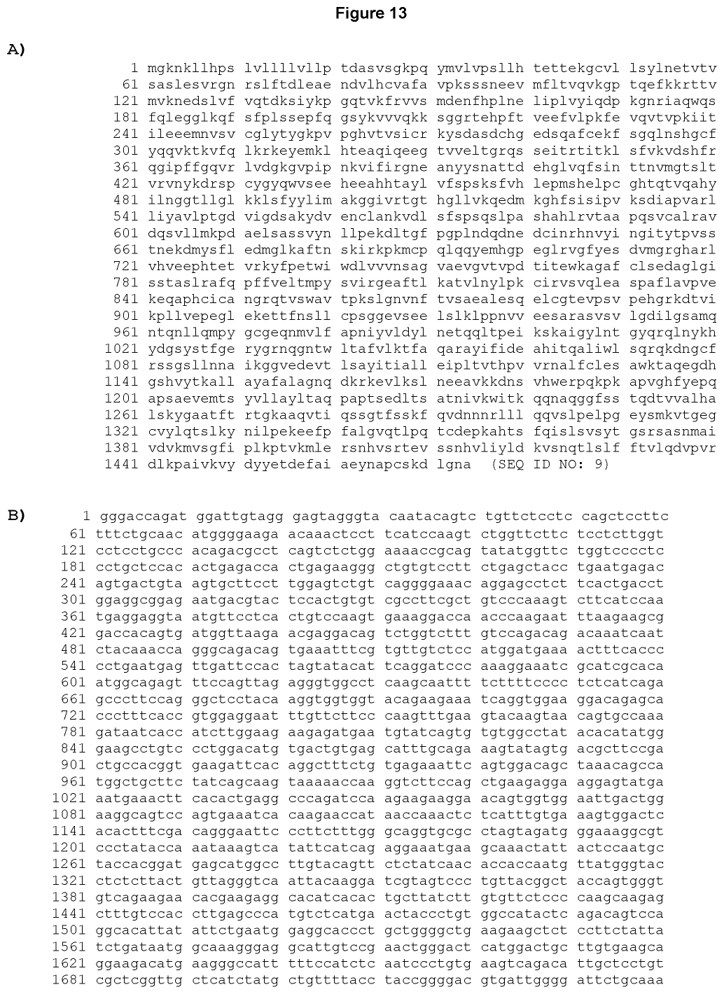

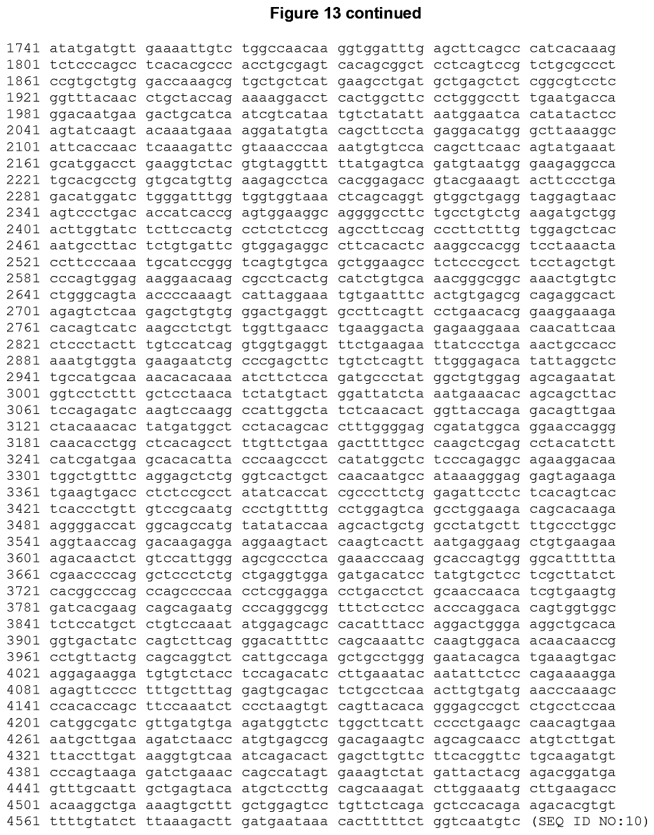

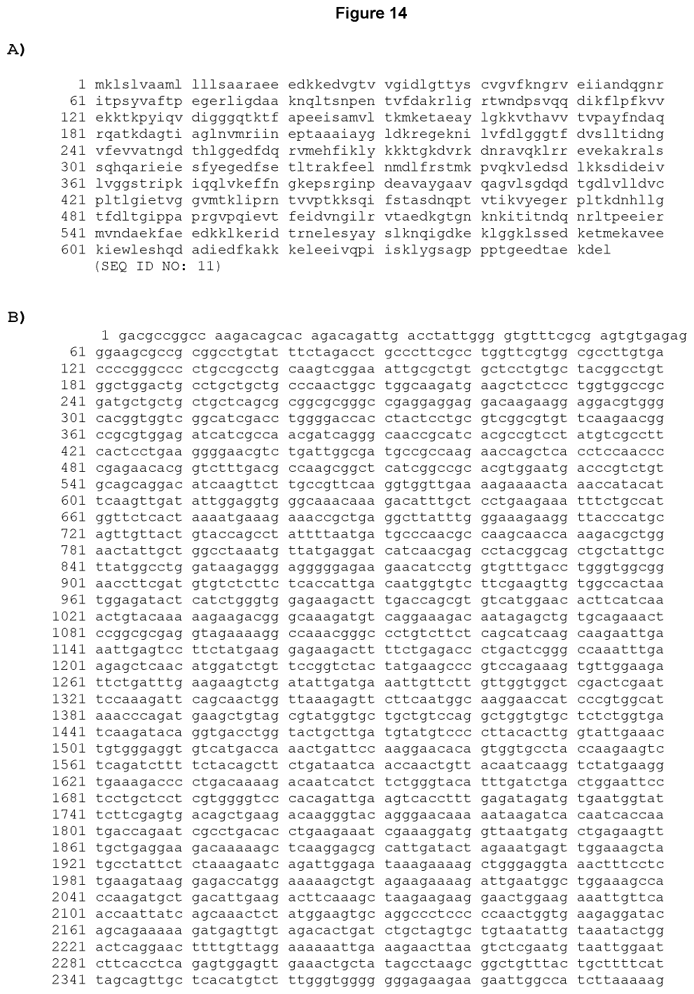

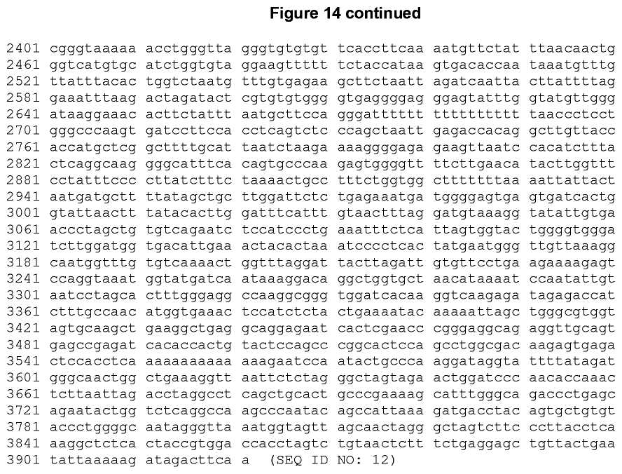

In one embodiment, gene expression of α2M or csGRP78 may be inhibited utilizing polynucleotides based on anti-sense or RNA interference inhibitors, e.g. siRNA, shRNA and the like which are derived from α2M- or csGRP78-encoding nucleic acid sequences such as the sequences shown in FIGS. 13B and 14B, respectively. In one embodiment, such polynucleotides may be used to inhibit upregulation of α2M which is associated with fibrosis, such as local upregulation of α2M in an organ (e.g. the kidney) in which fibrosis may develop or has developed.

Knowledge of the α2M and csGRP78 encoding nucleic acid sequences can be used to prepare antisense oligonucleotides effective to result in the desired inhibition. Such sequences, thus, may be used to prepare antisense oligonucleotides effective to bind to α2M or csGRP78 nucleic acid and inhibit the expression thereof. The term “antisense oligonucleotide” as used herein means a nucleotide sequence that is complementary to at least a portion of a target nucleic acid sequence. The term “oligonucleotide” refers to an oligomer or polymer of nucleotide or nucleoside monomers consisting of naturally occurring bases, sugars, and intersugar (backbone) linkages. The term also includes modified or substituted oligomers comprising non-naturally occurring monomers or portions thereof, which function similarly. Such modified or substituted oligonucleotides may be preferred over naturally occurring forms because of properties such as enhanced cellular uptake, or increased stability in the presence of nucleases. The term also includes chimeric oligonucleotides which contain two or more chemically distinct regions. For example, chimeric oligonucleotides may contain at least one region of modified nucleotides that confer beneficial properties (e.g. increased nuclease resistance, increased uptake into cells) as well as the antisense binding region. In addition, two or more antisense oligonucleotides may be linked to form a chimeric oligonucleotide.

The antisense oligonucleotides of the present invention may be ribonucleic or deoxyribonucleic acids and may contain naturally occurring bases including adenine, guanine, cytosine, thymidine and uracil. The oligonucleotides may also contain modified bases such as xanthine, hypoxanthine, 2-aminoadenine, 6-methyl, 2-propyl and other alkyl adenines, 5-halo uracil, 5-halo cytosine, 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thiolalkyl guanines, 8-hydrodyl guanine and other 8-substituted guanines, other aza and deaza uracils, thymidines, cytosines, adenines, or guanines, 5-tri-fluoromethyl uracil and 5-trifluoro cytosine.

Other antisense oligonucleotides of the invention may contain modified phosphorous, oxygen heteroatoms in the phosphate backbone, short chain alkyl or cycloalkyl intersugar linkages or short chain heteroatomic or heterocyclic intersugar linkages. For example, the antisense oligonucleotides may contain phosphorothioates, phosphotriesters, methyl phosphonates and phosphorodithioates. In addition, the antisense oligonucleotides may contain a combination of linkages, for example, phosphorothioate bonds may link only the four to six 3′-terminal bases, may link all the nucleotides or may link only 1 pair of bases.

The antisense oligonucleotides of the invention may also comprise nucleotide analogs that may be better suited as therapeutic or experimental reagents. An example of an oligonucleotide analogue is a peptide nucleic acid (PNA) in which the deoxribose (or ribose) phosphate backbone in the DNA (or RNA), is replaced with a polymide backbone which is similar to that found in peptides (P. E. Nielson, et al Science 1991, 254, 1497). PNA analogues have been shown to be resistant to degradation by enzymes and to have extended lives in vivo and in vitro. PNAs also form stronger bonds with a complementary DNA sequence due to the lack of charge repulsion between the PNA strand and the DNA strand. Other oligonucleotide analogues may contain nucleotides having polymer backbones, cyclic backbones, or acyclic backbones. For example, the nucleotides may have morpholino backbone structures (U.S. Pat. No. 5,034,506). Oligonucleotide analogues may also contain groups such as reporter groups, protective groups and groups for improving the pharmacokinetic properties of the oligonucleotide. Antisense oligonucleotides may also incorporate sugar mimetics as will be appreciated by one of skill in the art.

Antisense nucleic acid molecules may be constructed using chemical synthesis and enzymatic ligation reactions using procedures known in the art based on a given α2M or csGRP78 nucleic acid sequence such as that provided herein. The antisense nucleic acid molecules of the invention, or fragments thereof, may be chemically synthesized using naturally occurring nucleotides or variously modified nucleotides designed to increase the biological stability of the molecules or to increase the physical stability of the duplex formed with mRNA or the native gene, e.g. phosphorothioate derivatives and acridine substituted nucleotides. The antisense sequences may also be produced biologically. In this case, an antisense encoding nucleic acid is incorporated within an expression vector that is then introduced into cells in the form of a recombinant plasmid, phagemid or attenuated virus in which antisense sequences are produced under the control of a high efficiency regulatory region, the activity of which may be determined by the cell type into which the vector is introduced.

In another embodiment, siRNA/shRNA technology may be applied to inhibit expression of α2M or csGRP78. Application of nucleic acid fragments such as siRNA/shRNA fragments that correspond with regions in α2M or csGRP78 gene and which selectively target the gene may be used to block α2M or csGRP78 expression. Such blocking occurs when the siRNA/shRNA fragments bind to the gene thereby preventing translation of the gene to yield functional α2M or csGRP78.

SiRNA (small interfering RNA molecules) and shRNA (small hairpin RNA molecules), corresponding to α2M or csGRP78 are made using well-established methods of nucleic acid syntheses as outlined above with respect to antisense oligonucleotides. Since the structure of target α2M and csGRP78 genes are known, fragments of RNA that correspond therewith can readily be made.

It will be appreciated by one of skill in the art that siRNA/shRNA fragments useful in the present method may be derived from specific regions of the α2M or csGRP78-encoding nucleic acid which may provide more effective inhibition of gene expression. In addition, as one of skill in the art will appreciate, useful siRNA fragments need not correspond exactly with the α2M or csGRP78 target gene, but may incorporate sequence modifications, for example, addition, deletion or substitution of one or more of the nucleotide bases therein, provided that the modified siRNA retains the ability to bind selectively to the target gene. Selected siRNA fragments may additionally be modified in order to yield fragments that are more desirable for use. For example, siRNA fragments may be modified to attain increased stability in a manner similar to that described for antisense oligonucleotides.

The efficiency of selected polynucleotides to block α2M or csGRP78 activity can be confirmed using a cell-based assay, i.e. using cells that express α2M or csGRP78. Briefly, a selected polynucleodide may be incubated with the cell line under appropriate growth conditions. Following a sufficient reaction time, i.e. for the polynucleotide to bind with α2M or csGRP78 nucleic acid (DNA/mRNA) to result in decreased levels of free α2M or csGRP78 mRNA, the reaction mixture is tested to determine if such a decrease has occurred. Suitable polynucleotides will prevent processing of the α2M or csGRP78 gene to yield functional protein. This can be detected by assaying for relevant activity in a cell-based assay, e.g. inhibition of Akt activation, or reduction in extracellular matrix protein expression.

Once prepared, oligonucleotides determined to be useful to inhibit α2M or csGRP78 gene expression, such as antisense oligonucleotides and siRNA, may be used in the present therapeutic method to prevent or minimize the interaction of α2M and csGRP78. A suitable oligonucleotide may be introduced into tissues or cells of the mammal using techniques in the art including vectors (retroviral vectors, adenoviral vectors and DNA virus vectors) or by using physical techniques such as microinjection.

The interaction of α2M* and csGRP78 may also be inhibited at the protein level, for example, using immunological inhibitors, synthetic small molecules or peptide mimetics, for example, based on the binding sites of one or the other of α2M* or csGRP78.

Immunological inhibitors such as polyclonal and monoclonal antibodies that bind to α2M and prevent the activation of α2M*, and/or antibodies that bind α2M* or csGRP78 and which result in blocking of the interaction between α2M* and csGRP78 may be prepared using well-established hybridoma technology developed by Kohler and Milstein (Nature 256, 495-497 (1975)). Hybridoma cells can be screened immunochemically for production of antibodies specifically reactive with the region of α2M* or csGRP78 which is involved in their interaction, and the reactive monoclonal antibodies can then be isolated. Alpha-2M and GRP78 antibodies are also commercially available, for example, from Abcam, Proteintech and ThermoFisher Scientific. The term “antibody” as used herein is intended to include antigenic fragments thereof which retain the ability to specifically react with α2M* or csGRP78 according to the invention, as well as antigenic chimeric antibody derivatives, i.e., antibody molecules resulting from the combination of a variable non-human animal peptide region and a constant human peptide region

Peptide inhibitors may also be used to block the interaction between α2M* and csGRP78, for example, peptides designed to interact with the binding region of α2M* or the binding region of csGRP78. The receptor-binding domain of α2M* is in the C-terminus of α2M, residues 1314-1451 in the human sequence, in which the lysine residue at position 1374 is critical to binding to csGRP78, and the receptor-binding domain of GRP78 is the N-terminal region thereof from amino acids 98 to 115.

In one embodiment, the peptide inhibitor is based on the native csGRP78 binding region, namely, the region comprising amino acids from position 98 to 115 of csGRP78. A suitable peptide based on this region will comprise an N-terminal cysteine residue at residue position 97, and will retain the tertiary structure of this region in csGRP78. Preferably, the peptide comprises at least about 10 consecutive amino acid residues from this region, and may be about 10-50 amino acids in length, such as 10-25 amino acid residues in length. An example of a peptide inhibitor based on this region is the peptide, CLIGRTWNDPSVQQDIKFL (SEQ ID NO: 1). As one of skill in the art will appreciate, the peptide may include sequence modifications which do not adversely affect its function to block the interaction between α2M* and csGRP78. Sequence modifications may include one or more amino acid substitutions, deletions or additions. With respect to substitutions, preferably these are conservative amino acid substitutions with amino acids which are similar in structure and charge. Preferably, the peptide inhibitor retains at least about 75% sequence identity with the native csGRP78 binding region (i.e. includes no more than 5 amino acid substitutions, additions or deletions), and more preferably at least about 80%, 85%, 90% or 95% sequence identity (i.e. includes 4 or less sequence modifications). In one embodiment, one or more of the amino acids may be substituted with a D-amino acid analog, beta amino acid, cyclic/constrained amino acid or other unnatural amino acid. Amino acid sidechain modifications may also be incorporated within the peptide, such as incorporation of covalent reactive warheads, i.e. reactive groups which function to engage the target (α2M*) via covalent bond formation, e.g. reactive groups such as acrylamides, ester derivatives, α,β-unsaturated carbonyl compounds, α,β-unsaturated amides and sulfonyl fluoride derivatives.

The peptide may comprise one or more chemical modifications which stabilize the peptide, provide protease resistance (e.g. against any one or more of pepsin, trypsin, chymotrypsin, serum proteases or intracellular proteases) and/or favourably modify the pharmacokinetics of the peptide. Such modifications may include phosphorylation, glycosylation, and/or lipidation such as cysteine prenylation, N-terminal glycine myristoylation, cysteine palmitoylation, and serine and lysine fatty acylation. The peptide may be modified at its termini to prevent undesirable enzymatic or chemical degradation, including N-terminal and/or C-terminal blocking groups. Suitable N-terminal protecting groups include, for example, lower alkanoyl groups of the formula RC(O)— wherein R is a linear or branched C₁₋₅ alkyl chain. A preferred group for protecting the N-terminal end of the present compounds is the acetyl group, CH₃—C(O). Also suitable as N-terminal protecting groups are amino acid analogues lacking the amino function. Suitable C-terminal protecting groups include groups which form ketones or amides at the carbon atom of the C-terminal carboxyl, or groups which form esters at the oxygen atom of the carboxyl. Ketone and ester-forming groups include alkyl groups, particularly branched or unbranched C₁₋₅ alkyl groups, e.g. methyl, ethyl and propyl groups, while amide-forming groups include amino functions such as primary amines (—NH₂), or alkylamino functions, e.g. mono-C₁₋₅ alkylamino and di-C₁₋₅ alkylamino groups such as methylamino, ethylamino, dimethylamino, diethylamino, methylethylamino and the like. Amino acid analogues are also suitable for protecting the C-terminal end of the present compounds, for example, decarboxylated amino acid analogues such as agmatine.

The peptide may also be modified to incorporate a ligand that binds serum panel-reactive IgG antibodies to enhance serum stability and peptide circulation time. Such a ligand may be positioned anywhere on the peptide sequence. The ligand will generally be a cell surface antigen such as leukocyte antigen (HLA), peptidyl-glycine alpha-amidating monooxygenase (PAM), Fc-III, FcBP-1, FcBP-2, Fc-III-4c and FcRM.

Candidate inhibitors may be screened for inhibitory activity by assaying for relevant activity in a cell-based system as described above for the determination of inhibitory polynucleotides. In this case, cells are incubated with the candidate inhibitor and will be monitored for direct inhibition of the interaction between α2M* and csGRP78. This inhibition is similarly detected by assaying for relevant activity in the cell-based assay, e.g. inhibition of Akt activation, or reduction in extracellular matrix protein expression.

The present anti-fibrotic inhibitor may be modified to augment fibrotic organ targeting. For example, the inhibitor may be conjugated to an entity that targets fibrotic tissue or targets a particular organ (e.g. lung, kidney or liver), such as an antibody or region thereof that targets a protein expressed by fibrotic tissue. An example of such an entity is an antibody or fragment thereof (variable domain) that targets the fibronectin EDA isoform (FnEDA) expressed in fibrotic kidney. An example of a modification that targets a particular organ is conjugation of the inhibitor to a ligand that selectively targets cells of the target organ. Examples include: 1) conjugation of galactose to the inhibitor to target hepatocytes, the primary cause of liver fibrosis, since galactose is a specific ligand for the asialoglycoprotein receptor (ASGP-R) located primarily on the surface of hepatocytes: 2) conjugation to a kidney-targeting peptide (LPVAS (SEQ ID NO: 2)) which targets several cell types in the kidney: 3) conjugation to a low-molecular-weight chitosan (LMWC)-zinc complex shown to efficiently deliver drug to kidney.

Further, the anti-fibrotic inhibitor may be packaged in a drug delivery system that targets fibrotic tissue. In one embodiment, a macrophage-mediated drug delivery system is provided for use to deliver the anti-fibrotic inhibitor to the lung. Macrophage-mediated drug delivery systems can be prepared by loading inhibitor or nanoparticles loaded with the inhibitor, such as liposomes, gold and/or silica nanoshells, graphene nanocrystals, chitosan albumin or other polymer nanoshells, into macrophages, macrophage membranes or macrophage-derived vesicles. The macrophages may be primary macrophages such as bone marrow-derived macrophages, alveolar macrophages or peritoneal macrophages or macrophages cultured in cell banks.

A therapeutic anti-fibrotic inhibitor in accordance with the invention may be administered to a subject to block the interaction between α2M* and csGRP78, to treat fibrosis in the subject. The term “treat”, “treating” or “treatment” is used herein to refer to methods that favorably alter a pathological fibrotic condition, including those that moderate, reverse, reduce the severity of, or protect against, fibrosis in a subject. While not wishing to be limited to any particular theory, blocking the interaction between α2M* and csGRP78 inhibit profibrotic Akt activation to result in inhibition of downstream matrix and profibrotic cytokine production. The term “subject” as used herein refers to mammalian subjects, including both human and non-human mammals. The inhibitor is administered to a subject in a therapeutically effective amount. The term “therapeutically effective amount” is an amount of the inhibitor required to treat fibrosis, for example, reduce the production of extracellular matrix protein by at least about 10% or more, such as by at least 20%, 30%, 40%, 50%, 60%, 70%, 80%, 90% or more. Dosages of the anti-fibrotic inhibitor that are therapeutically effective will vary on many factors including the condition and severity of the condition to be treated, the nature of the inhibitor, as well as the particular individual being treated. Appropriate dosages of a peptide-based inhibitor may in the range of about 1 ng/kg to about 1 mg/kg, based on its pharmacokinetic properties. Dosages of an antibody-based inhibitor may range from 10 mg to 1500 mg, or individualized dosing may be established based on point-of-care assessment of antibody concentrations.

A therapeutic anti-fibrotic inhibitor, including nucleic acid-based, protein-based and other inhibitors, may be administered in combination with a suitable pharmaceutically acceptable carrier. The expression “pharmaceutically acceptable” means acceptable for use in the pharmaceutical and veterinary arts, i.e. not being unacceptably toxic or otherwise unsuitable. Examples of pharmaceutically acceptable carriers include diluents, excipients and the like. Reference may be made to “Remington's: The Science and Practice of Pharmacy”, 21st Ed., Lippincott Williams & Wilkins, 2005, for guidance on drug formulations generally. The selection of adjuvant depends on the type of inhibitor and the intended mode of administration of the composition. In one embodiment of the invention, the compounds are formulated for administration by infusion, or by injection either subcutaneously, intravenously, intradermal, intramuscular, intraperitoneal intrathecally, intraspinally, epicutaneously or as part of an artificial matrix, and are accordingly utilized as aqueous solutions in sterile and pyrogen-free form and optionally buffered or made isotonic. Thus, the compounds may be administered in distilled water or, more desirably, in saline, phosphate-buffered saline or 5% dextrose solution. Compositions for oral administration via tablet, capsule or suspension are prepared using adjuvants including sugars, such as lactose, glucose and sucrose: starches such as corn starch and potato starch; cellulose and derivatives thereof, including sodium carboxymethylcellulose, ethylcellulose and cellulose acetates: powdered tragancanth: malt: gelatin: talc: stearic acids: magnesium stearate: calcium sulfate: vegetable oils, such as peanut oils, cotton seed oil, sesame oil, olive oil and corn oil: polyols such as propylene glycol, glycerine, sorbital, mannitol and polyethylene glycol: agar: alginic acids: water: isotonic saline and phosphate buffer solutions. Wetting agents, lubricants such as sodium lauryl sulfate, stabilizers, tableting agents, anti-oxidants, preservatives, colouring agents and flavouring agents may also be present. Aerosol formulations, for example, for nasal delivery, may also be prepared in which suitable propellant adjuvants are used. Other adjuvants may also be added to the composition regardless of how it is to be administered, for example, anti-microbial agents may be added to the composition to prevent microbial growth over prolonged storage periods.

The present therapeutic inhibitor may be administered to a subject in conjunction with one or more other therapeutic agents to enhance the treatment of fibrosis. For example, the inhibitor may be co-administered with another drug useful to treat fibrosis, either in a combined composition, or in separate compositions administered at the same or different times. In one embodiment, for the treatment of kidney fibrosis, the present anti-fibrotic inhibitor may be used in conjunction with an incretin hormone such as glucagon-like peptide-1 (GLP-1) receptor agonist or glucose-dependent insulinotropic peptide (GIP), which promotes insulin release from the pancreas. Examples include Exenatide, Semaglutide and Dulaglutide, which are administered by injection, or orally (Semaglutide).

In another embodiment, an orally administrable antifibrotic inhibitor may be administered in combination with an orally administrable anti-diabetes medication, such as metformin, an SGLT2 inhibitor, sulfonylurea, or orally bioavailable peptides such as N-acetyl-seryl-aspartyl-lysyl-proline or AcSDKP (SEQ ID NO: 3)

### I. DEFINITIONS

Unless otherwise indicated, the definitions and embodiments described in this and other sections are intended to be applicable to all embodiments and aspects of the present application herein described for which they are suitable as would be understood by a person skilled in the art.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

In embodiments comprising an “additional” or “second” component, such as an additional or second peptide, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

It is apparent that various modifications can be made to the peptide of the invention. Said modifications include but are not limited to: hydroxylation of proline and lysine, methylation of o-amino group on the side chain of lysine or histidine, acetylation of N-terminal amino, amidation of C-terminal carboxyl, replacement with D amino acid analogues, creation of disulfide-bridged peptide dimer, addition of covalent warhead to amino acid side chain, head to tail cyclization, amino acid capping to promote helicity, and use of unnatural amino acids based on structure-activity relationship analysis. These modified polypeptides which comprises the disclosed amino acid sequences are also encompassed within the scope of the present invention.

The following example illustrates the scope of the invention. Specific elements of the example are for descriptive purposes only and are not intended to limit the scope of the invention. Those skilled in the art could develop equivalent methods and utilize comparable materials that are within the scope of the invention.

### Example

Cell Culture-Primary mesangial cells (MC) were obtained from male C57BL/6 mice by glomerular sieving and cultured in Dulbecco's modified Eagle's medium (Sigma) supplemented with 20% fetal bovine serum, streptomycin (100 μg/mL) and penicillin (100 μg/mL). 1LN prostate cancer cells, which express high levels of csGRP78 (as described in Hart et al., J Biol Chem. 2002; 277 (44): 42082-42087) were cultured in RPMI 1640 supplemented with 10% fetal bovine serum (ThermoFisher). Cells were grown at 37° C. in 95% 02 and 5% CO₂. The immortalized human proximal tubular cell line HK2 (ATCC, Burlington, Canada) was cultured in 10% FBS in DMEM/F12 medium. Primary rat renal fibroblasts (Cell Biologics, Burlington, Canada) and cultured in 10% FBS in DMEM/F12 medium. MC were serum deprived in medium with 1% bovine serum albumin (BSA) and HK2 and RF cells were starved in 0.5% FBS 24 h before treatment with HG (30 mM), mannitol (24.4 mM) as osmotic control or methylamine-activated α2M (100 pM). The peptide sequence in GRP78 to which α2M* binds was used to block their interaction (CLIGRTWNDPSVQQDIKFL) (Leu⁹⁸-Leu¹¹⁵ with an additional N-terminal cysteine) (Gopal et al., J Biol Chem. 2016; 291 (20): 10904-10915). The scrambled peptide, GTNKSQDLWIPQLRDVFI (SEQ ID NO: 4) was used as a control, with both peptides used at 100 nM (Cedarlane). Additional peptides were synthesized by Biomatik as follows: 1) peptide lacking the N-terminal cysteine, 2) peptide in which the cysteine was replaced by a methionine, 3) dimerized peptide via the cysteine (LFKIDQQVSPDNWTRGILC—S—S— CLIGRTWNDPSVQQDIKFL) (SEQ ID NO: 5) head to tail cyclization via an amide which leaves the cysteine intact.

Protein Extraction and Immunoblotting-Cells were lysed as described previously (Krepinsky et al. Nitric Oxide Inhibits Stretch-Induced MAPK Activation in Mesangial Cells Through RhoA Inactivation. Published online 2003. doi: 10.1097/01.ASN.00000940.85.04161.A7). Proteins were separated using SDS-PAGE followed by immunoblotting. Antibodies used were: α2M (1:1000, Bioss), mouse monoclonal F-α2M, which detects only conformationally changed α2M* as previously described (1:1000), pAkt S473 (1:1000, Cell Signaling), total Akt (1:1000, Cell Signaling),), pSmad3 Ser423/425 (1:4000, Novus), total Smad3 (1:1000, Abcam), phosphorylated focal adhesion kinase (pFAK) Tyr-397 (1:1000, Millipore, 07-012), FAK (1:1000, Santa Cruz Biotechnology, SC558), LRP1 (1:1000, Abcam), GRP78 (C20) (1:1000, Santa Cruz), platelet derived growth factor-β (PDGFR-β) (1:1000, Cedarlane), collagen IV (Col IV) (1:1000, Cell Signaling), fibronectin (FN) (1:1000, Abcam), connective tissue growth factor (CTGF) (1:1000, Santa Cruz), and tubulin (1:2000, Santa Cruz).

Media was concentrated using a 156 kDa column (Amicon Ultra 4 mL Centrifugal Filter) and run on a non-denaturing polyacrylamide gel. After transfer, membranes were probed for both inactive α2M and the conformationally changed and more rapidly migrating α2M *. Nativemark unstained protein ladder (ThermoFisher) and methylamine-activated α2M were used to confirm band location.

RNA Analysis Using qPCR-RNA was extracted using Trizol (Invitrogen), and 1 μg was reverse transcribed using qScript Supermix Reagent (Quanta Biosciences). Primers for α2M were as follows: forward 5′-CCAGGACACGAAGAAGG-3′ (SEQ ID NO: 6) and reverse 5′-CACTTCACGATGAGCAT-3′ (SEQ ID NO: 7). Quantitative PCR was performed using the Power SYBR Green PCR Master Mix on the Applied Biosystems Vii 7 Real-Time PCR System. Changes in mRNA expression were determined relative to 18S using the ΔΔCt method.

Experimental Animals and Tissue Processing-All studies were conducted in accordance with McMaster University and the Canadian Council on Animal Care guidelines. Two type 1 diabetic models were assessed: 1) Male type 1 diabetic C57BL/6-Ins2^{Akita}/J mice (Jackson Laboratories) and their wild-type controls were sacrificed at 18, 30, and 40 weeks of age. 2) Male CDI mice (Charles River) were nephrectomized, followed by injection with 200 μg streptozotocin and sacrifice after 12 weeks of diabetes. For human studies, kidney biopsy samples with a diagnosis of DKD were obtained. Normal kidney tissues surrounding resected renal cancers were used as controls. Tissue was obtained after approval by the local Research Ethics Board (REB). The db/db type 2 diabetic model was also used, with kidney tissue from 16-week old male diabetic or control mice obtained from a collaborator. To generate the non-diabetic CKD model, male CDI mice (Charles River) underwent a ⅚ nephrectomy (Nx) done in 2 stages (uninephrectomy followed by ⅔ resection of the contralateral kidney a week later), with kidney assessment 9 weeks following the second surgery. Lastly, a mouse model for IPF was generated by intratracheal injection of bleomycin under anesthesia, with lungs harvested 21 days following injection.

For immunoblotting, samples were homogenized in tissue lysis buffer containing protease inhibitors (complete Mini, Sigma and PhosSTOP, Sigma) in the Bead Mill Homogenizer (Bead Ruptor Elite, Omni International) using 1.4 mm ceramic beads (Lysing Matrix D, MP Biomedicals). After clarification of lysate by centrifugation, protein concentration was determined using the DC Protein Assay (Bio-Rad).

For immunohistochemistry (IHC), paraffin embedded kidney sections were cut at 4 μm, deparaffinized and then probed for α2M (Bioss, 1:1000, no antigen retrieval) or α2M* (Fα2M antibody (see above, 1:100, antigen retrieval using proteinase K, 40 μg/ml, 5 min). Images were taken at 20× and 40× and quantified using Image J software.

For immunofluorescence (IF), kidney sections preserved in OCT were cut at 10 μm, fixed with 3.7% paraformaldehyde and permeabilized with 0.2% Triton X-100. To block endogenous biotin and reduce high background fluorescence, an Avidin/Biotin Blocking Kit (Vector Labs) was used (15 min as per manufacturer's protocol), followed by co-staining with Fα2M (1:200) and α8-integrin as a mesangial cell marker (1:100, Novus Biologicals). For cell surface colocalization of csGRP78 and the plasma cell surface marker wheat germ agglutinate (WGA), tissues were not fixed or permeabilized prior to staining. Slides were incubated with csGRP78 (1:50,000) overnight and the following day with WGA (1:400) for 20 minutes. Images were captured using the Olympus BX41 microscope at 40×. The Image J colocalization plug-in was used to create a colocalization mask of areas expressing both α8-integrin and Fα2M. This was then quantified using Image J.

For in situ hybridization (ISH), 4 μm paraffin embedded sections were deparaffinized using xylene and ethanol, fixed with 4% paraformaldehyde, and digested with proteinase K (20 mg/mL, 5 min). Slides were pre-hybridized in hybridization buffer (ultra pure 50% formamide, 20×SSC, 10 μg/μl yeast t-RNA, 50×Denhardt's solution) in a heated humidified chamber at 53° C. for 2 h, followed by incubation with a denatured custom DIG-labeled α2M probe (5′AAGTAGCTTCGTGTAGTCTCT3′ (SEQ ID NO: 8), Qiagen) for 2 days. Slides were then washed with 2×SSC at room temperature followed by washes with 2×SSC and 0.1×SSC at hybridization temperature. After blocking in 1× Casein, slides were incubated with AP-coupled anti-DIG antibody (Abcam) overnight at 4° C., then developed using NBT/BCIP (Vector Laboratories). The reaction was stopped with TE buffer (Tris 10 mM/EDTA 1 mM, pH 8.0), after which slides were dehydrated and mounted using Faramount aqueous mounting medium (Dakocytomation).

Transfection—For siRNA experiments, MC were plated at 50-60% confluence and transfected with 100 nmol of α2M, LRP1 or control siRNA (Silencer Select, ThermoFisher). After 18 h, media was changed, and the following day cells were serum deprived as above prior to treatment and harvesting for analysis.

Electroporation was used to transfect cells with pcDNA 3.1 GRP78 ΔKDEL (GRP78 lacking the KDEL domain which localizes it to the ER, thus enabling significant localization to the cell surface, generously provided by Dr. Jeffrey Dickhout (McMaster University, Canada). Empty vector was used as a control. MC were grown to 100% confluency, trypsinized, and centrifuged in medium with 20% FBS without antibiotics. Cells (200 μl, 5×10⁵/ml) were then placed in a 4 mm gap electroporation cuvette with 10 μg of plasmid and electroporated using the ECM-830 system (ECM 399, BTX Harvard Apparatus) for one 30 ms pulse at 250V. Cells were plated in full medium with no antibiotics until recovery. The following day, media was changed to full medium with antibiotics, and at 80% confluency cells were starved as above prior to treatment.

Intracellular Calcium Assay—1LN cells were plated in a 96 black walled clear bottom plate and allowed to grow to confluence over 2 days. After loading with the calcium indicator Fura-2AM (5 μM, Abcam) in HBSS for 45 minutes at 37° C. in the dark, baseline fluorescence readings were taken every minute for 5 minutes using a temperature-controlled fluorescent microplate reader (Gemini EM Spectra Max, Molecular Devices) set to 340 and 380 nm excitation and 510 nm emission. After treatment with methylamine-activated α2M (100 pM) with or without antibody (Fα2M or control IgG, 2 μg) and with or without peptide or scrambled peptide (100 nM), readings were taken every minute for 15 minutes. Intracellular calcium concentrations were determined by calculating the ratio of fluorescence signal (340/380 nm).

Biotinylation for Cell Surface Protein Isolation—After treatment, cells were washed with cold phosphate-buffered saline (PBS), then incubated with 1 mg/ml EZ-linked Sulfo-Biotin (Pierce) for 30 minutes. They were then washed with 0.1 M glycine in PBS to remove excess Sulfo-Biotin, lysed in protein lysis buffer used above, clarified and equal quantities of protein incubated overnight in a 50% Neutravidin slurry (Fisher) to capture biotin-tagged proteins. Beads were then washed 5× with lysis buffer and bound proteins cleaved by boiling for 10 minutes in 2× PSB. Samples were separated using SDS-PAGE prior to immunoblotting.

TGFβ1 ELISA—MC media was collected after treatment and total secreted TGFβ1 was quantified using the TGFβ1 Quantikine ELISA Kit (R&D Systems).

Patient Cohort—The relationship between urinary α2M* and urinary TGFβ1 was studied using samples from a published cohort of type 2 diabetic patients with overt DKD who previously participated in a longitudinal biomarker study (Verhave et al., Diabetes Res Clin Pract. 2013; 101 (3): 333-340). All patient participation had been approved by clinic ethics committees and each patient gave informed consent to biobank urinary specimens for use at a subsequent time to test new hypotheses relevant to their disease. First, it was explored whether or not urinary α2M* was associated with total proteinuria in a sample from 4 subjects with proteinuria of <0.5 g/g creatinine and 4 with >2 g/g. Second, the association between α2M* and urinary TGFβ1 was verified in 18 subjects with proteinuria <2 g/g to attenuate the influence of proteinuria, which is also known to correlate with urinary TGFβ1. Patients had provided multiple urinary specimens during their follow-up and α2M* was determined for each available sample. Urine TGFβ1 was previously assessed by ELISA (Millipore) ²⁶. Urine α2M* was detected by ELISA using the Fα2M antibody as described previously ²³. Values were normalized to urine creatinine.

Statistical Analysis—Student's t-test or one-way ANOVA were used to compare the means between two or more groups, respectively. For calcium assay quantification, fold change at time of treatment (6 minutes) was compared between groups using a one-way ANOVA. Significant differences between multiple groups (post hoc) were analyzed using Tukey's HSD with p≤0.05 considered significant. Data are presented as mean±SEM. To assess the relationship between urinary biomarkers in patients with DKD, we used the Spearman Rho's or Pearson correlations depending on whether data had skewed or normal distributions.

### RESULTS

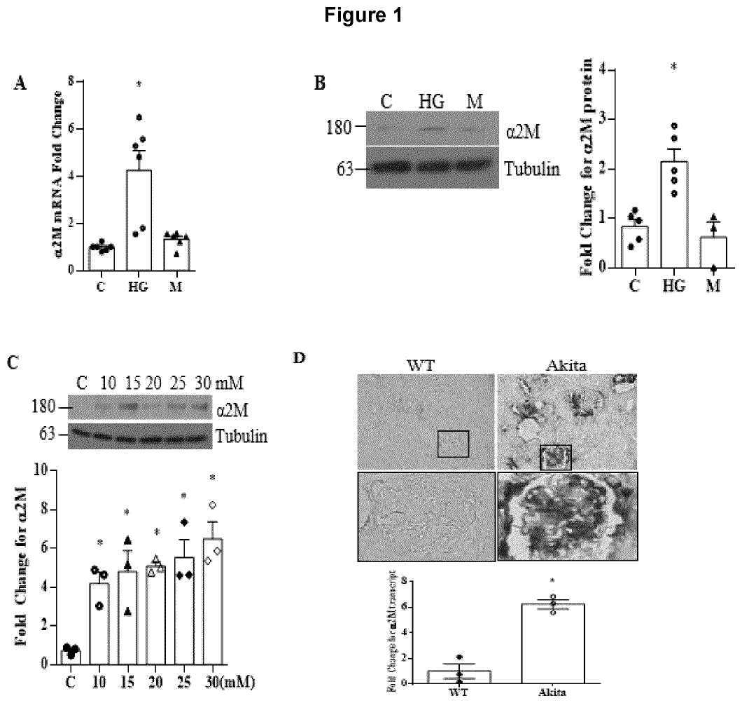

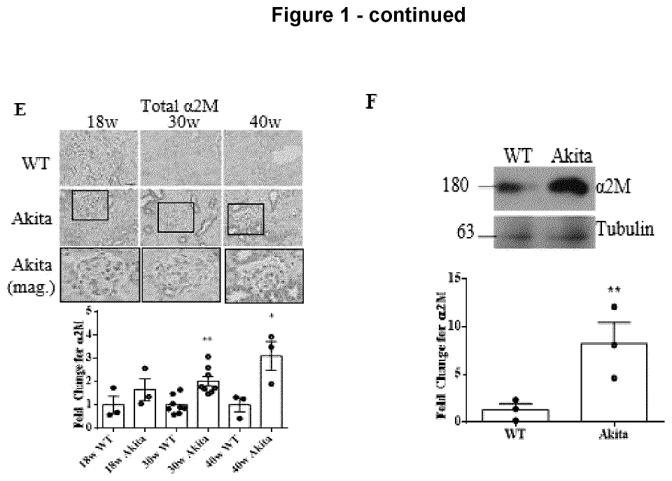

α2M is increased and activated by HG in MC and in diabetic kidneys—Previous reports have shown that α2M is increased in saliva and serum of diabetic patients and transcript levels were elevated in diabetic kidneys. Here it was first determined whether HG increased α2M transcript and protein expression in MC. FIGS. 1A and B show that HG, but not the osmotic control mannitol, increased both α2M mRNA and protein expression, respectively, and that this was dose dependent (FIG. 1C). Next, increased α2M expression in type 1 diabetic Akita kidneys by ISH was confirmed (FIG. 1D). In FIG. 1E, increased α2M protein levels were also seen by IHC in glomeruli as well as in some tubules. This was confirmed by immunoblotting of kidney lysates (FIG. 1F).

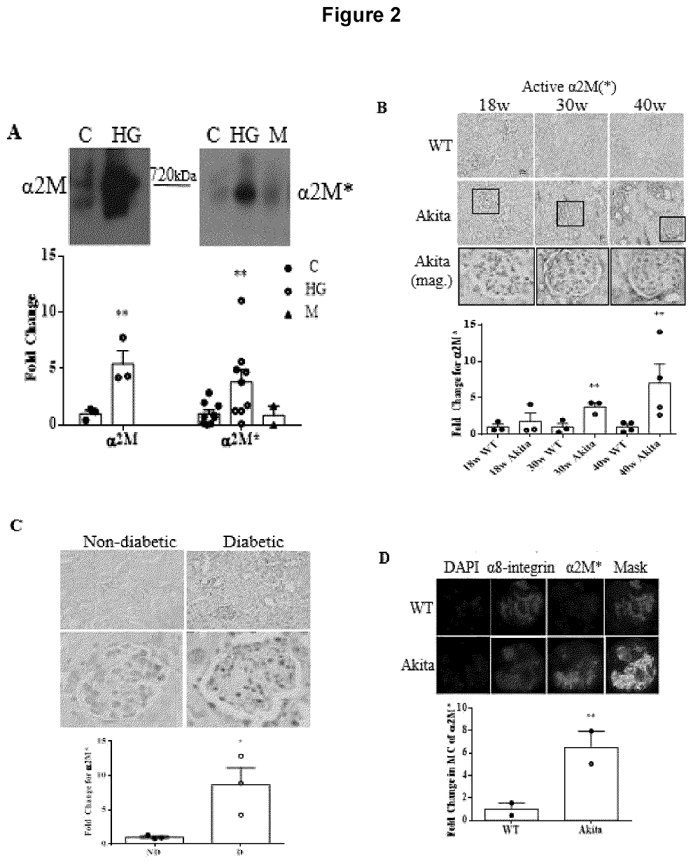

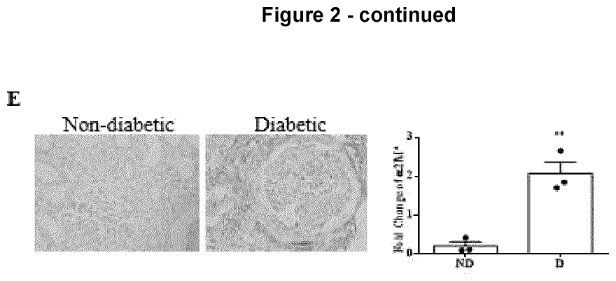

It was then determined whether α2M is also activated in HG, thereby revealing the binding site for csGRP78. Since activation entails conformational change, a non-denaturing gel was used to preserve α2M* tertiary structure. α2M* was detected with Fα2M, an antibody which specifically recognizes the revealed receptor binding domain in α2M*²³. FIG. 2A shows that both α2M and α2M* are significantly increased in the medium by HG, with α2M* unaffected by mannitol. As expected, α2M* migrated further through the gel than the inactive form. Then it was determined whether α2M* was increased in diabetic kidneys. As previously seen for α2M, α2M* was also elevated in both glomeruli and some tubules in Akita diabetic kidneys as shown by IHC (FIG. 2B). This was also seen in a second model of type 1 DKD (streptozotocin-treated uninephrectomized CDI mice). To confirm localization to MC, IF with dual staining was performed for α2M* and the MC marker α8-integrin in Akita diabetic kidneys. Using a colocalization mask, an increase in α2M* is seen in the mesangium (FIG. 2D). Finally, to determine whether α2M* is also increased in human DKD, its expression was assessed by IHC in kidney biopsies with a DKD diagnosis in comparison to normal kidney tissue taken at the time of renal cancer resection. FIG. 2E shows that α2M*, not seen in control kidneys, is seen in diabetic glomeruli. Taken together, these data confirm that α2M expression and activation are induced by HG.

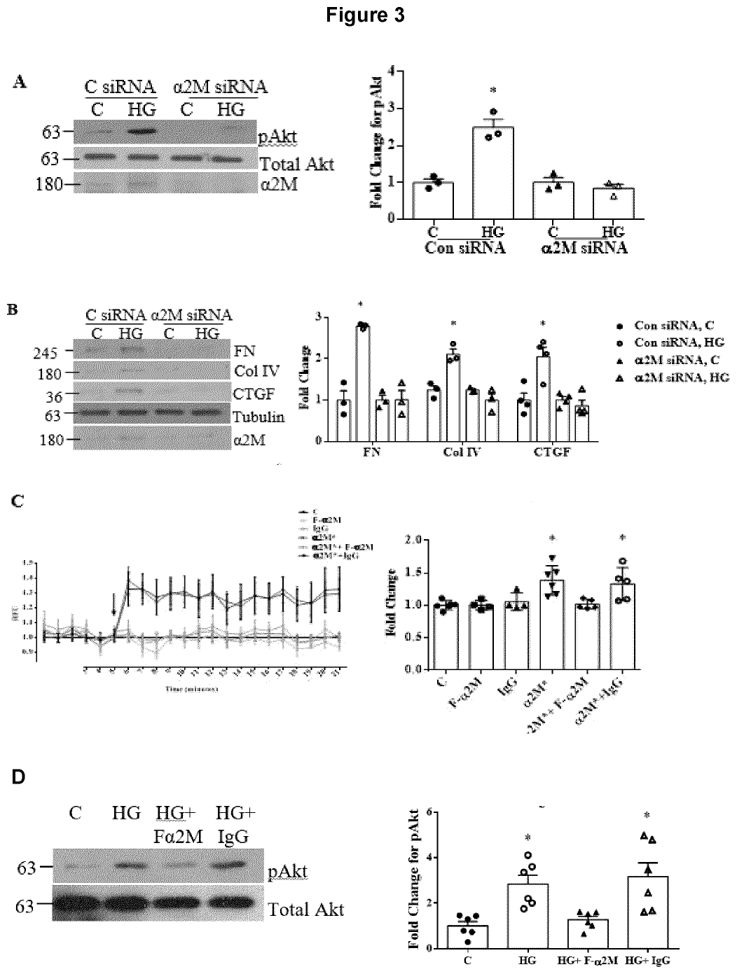

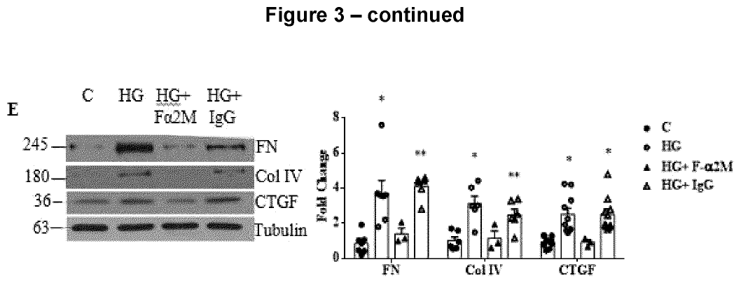

Inhibition of α2M α2M* inhibits HG-induced profibrotic responses by MC—Previous studies identified the importance of PI3K/Akt signaling in ECM protein production by MC in response to HG. As it was previously shown that csGRP78 mediates this signaling pathway, we wished to determine whether α2M* could be the ligand leading to its activation. We thus first investigated the effects of α2M downregulation using siRNA on HG-induced PI3K/Akt activation. As seen in FIG. 3A, α2M knockdown prevented activation of Akt, assessed by its phosphorylation on S473, in response to HG. Knockdown also significantly reduced ECM protein expression (fibronectin, collagen IV) and that of the profibrotic cytokine CTGF, known to contribute to mesangial expansion and kidney fibrosis in DKD²⁹ (FIG. 3B).

The requirement for α2M activation was then evaluated using the Fα2M antibody. As it binds the α2M receptor binding domain, which is exposed after activation and through which it binds to csGRP78, it was used here to functionally neutralize α2M* (Biltoft et al., Clin Biochem. 2017; 50 (18): 1203-1208). To confirm its neutralizing ability, 1LN prostate cancer cells were used which have high expression of csGRP78, and in which α2M* was shown to induce a rapid increase in intracellular calcium. FIG. 3C shows that Fα2M, but not control IgG, prevented this calcium rise, confirming its neutralizing ability. We then tested its effects in HG. As seen in FIGS. 3D and E, α2M* neutralization inhibited Akt activation and ECM/CTGF upregulation, similar to that observed with α2M knockdown.

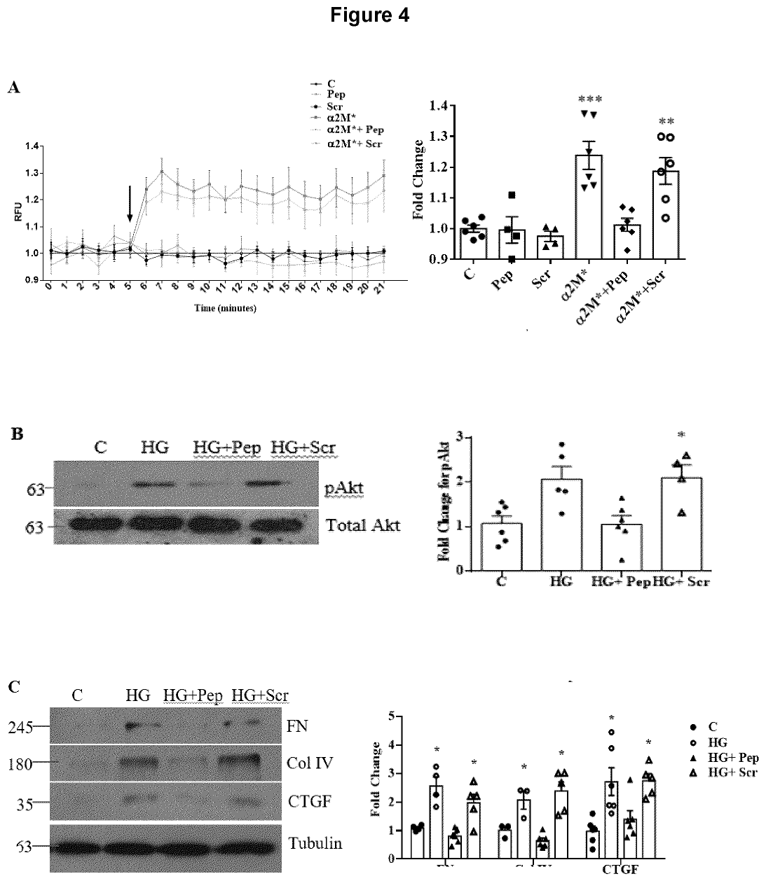

Previous studies showed that the receptor-binding domain of α2M* binds to the sequence Cys-Leu⁹⁸-Leu¹¹⁵ in GRP78 (Gopal et al. J Biol Chem. 2016: 291 (20): 10904-10915). Calcium signaling in 1LN cells induced by α2M* was abolished by a peptide comprising these residues. FIG. 4A confirms that this peptide, but not a scrambled control peptide, inhibits the α2M *-induced increase of intracellular calcium in 1LN cells. This peptide was used to confirm that α2M* mediates the HG-induced profibrotic response in MC. As seen in FIGS. 4B and C, this peptide abrogated HG-induced Akt activation and matrix protein/CTGF upregulation. Scrambled peptide had no effect. Taken together, these data support the importance of α2M and its activation in mediating the profibrotic response to HG, likely through csGRP78.

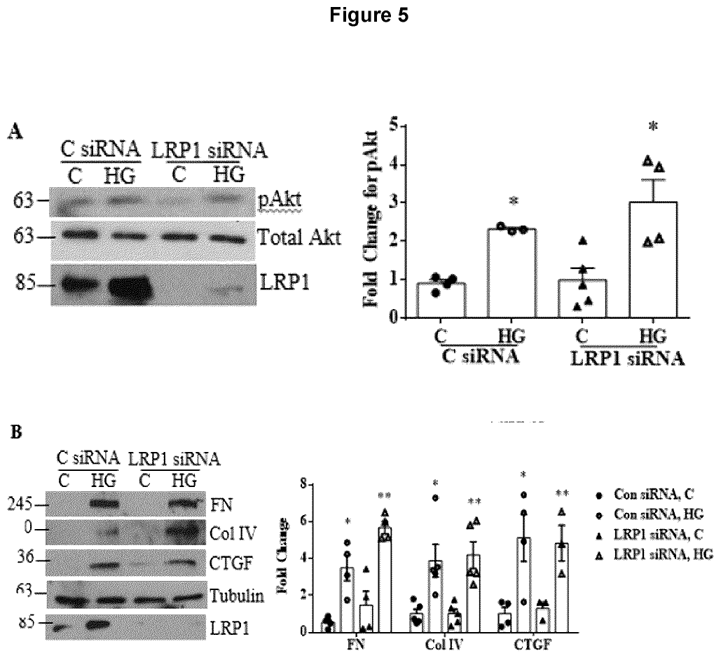

LRP1 is not involved in HG-induced profibrotic responses in MC—As described above, both LRP1 and csGRP78 are receptors for α2M*, although its affinity is significantly higher for csGRP78. To determine whether LRP1 is involved in mediating the HG response, the effect of LRP1 knockdown using siRNA was evaluated. As seen in FIGS. 5A and B, this did not affect HG-induced Akt activation or matrix/CTGF upregulation, indicating that csGRP78 rather than LRP1 mediates α2M* profibrotic signals in HG.

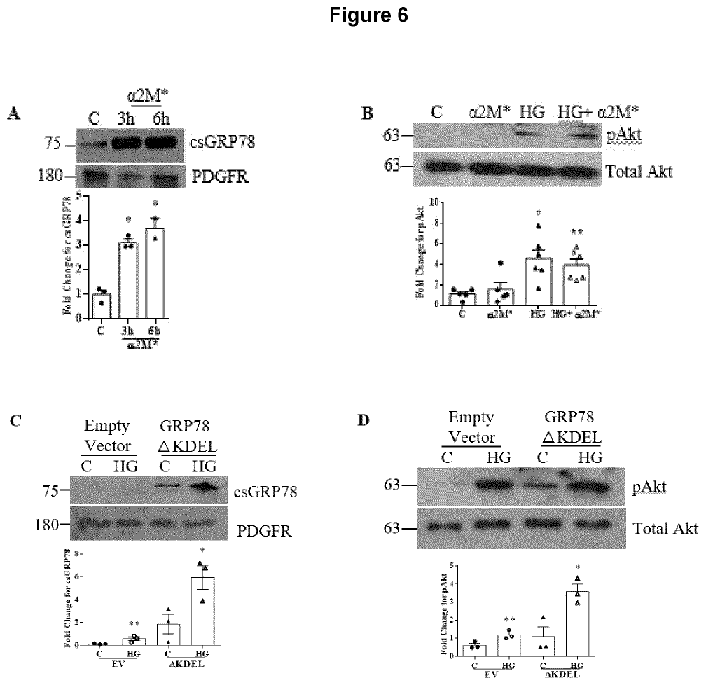

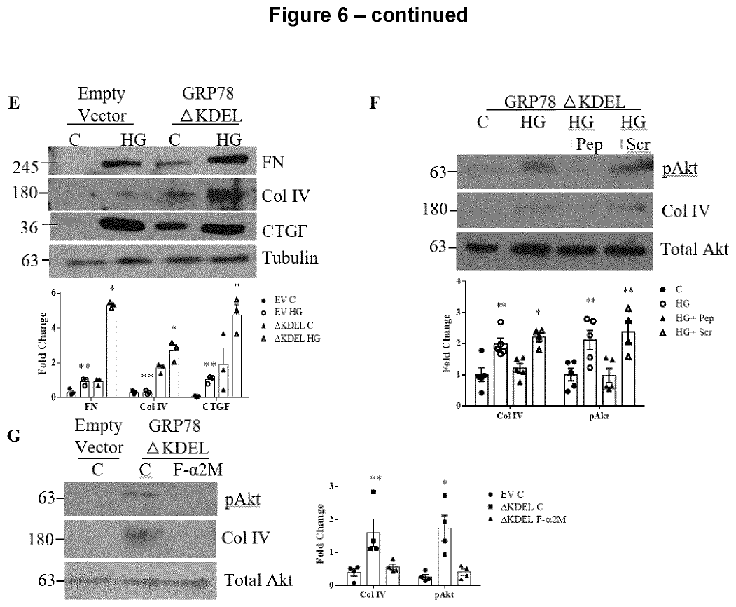

Increased matrix synthesis with csGRP78 overexpression requires α2M*—In prostate cancer cells, α2M* increased csGRP78. We tested whether this positive feedback loop also occurs in MC using a biotinylation assay to detect csGRP78. As shown in FIG. 6A, α2M* increased csGRP78 in the absence of HG. We further assessed whether HG-induced Akt activation could be augmented by α2M* cotreatment (FIG. 6B). However, no additive effect was seen, suggesting that the HG-induced increase in α2M* is sufficient to generate enough ligand to occupy available csGRP78.

Further, it was evaluated whether forced GRP78 translocation to the cell surface can initiate signaling or augment HG responses. Thus, GRP78 lacking the ER-retention signal KDEL (GRP78ΔKDEL) was overexpressed. First, it was confirmed by biotinylation and pull-down of cell surface proteins that its overexpression increases csGRP78 (FIG. 6C). Interestingly, a further increase was seen with HG. Next was examined its effects on Akt activation. As seen in FIG. 6D, overexpression itself led to Akt activation and this was further augmented by the addition of HG (FIG. 6D). Similar effects were seen for ECM protein accumulation and CTGF expression (FIG. 6E). These data suggest that in the absence of HG, basal levels of α2M* (as seen in FIG. 2A) induce signaling through csGRP78. To test this, the α2M* blocking peptide was used. As shown in FIG. 6F, this blocked Akt activation and matrix synthesis induced by csGRP78 overexpression. Similar results were seen with α2M* neutralization using the Fα2M antibody (FIG. 6G). Taken together, these data support a critical role for α2M* in MC profibrotic signaling.

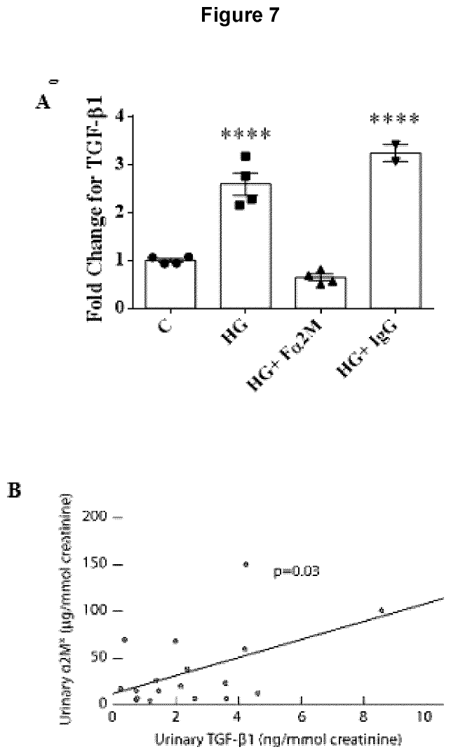

α2M* regulates TGFβ1 production by HG in MC—TGFβ1 is a major mediator of the profibrotic process in DKD and of HG-induced matrix upregulation in MC. Since Akt is known to regulate its synthesis in response to HG, inhibition of α2M* is likely to inhibit TGFβ1 production. Confirmation of this is shown in FIG. 7A, in which HG-induced secretion of TGFβ1 into the medium, assessed by ELISA, was blocked by α2M* neutralization with Fα2M.

Urinary α2M* is associated with urinary TGFβ1 in individuals with overt DKD—It was previously shown that urinary TGFβ1 contributed to prediction of kidney disease progression in a cohort of patients with established DKD (Verhave et al. 2013). Thus, the urine of a subset of these patients was analyzed to address the association between α2M* and TGFβ1. First, it was found in a subset of individuals with low and high proteinuria, as described in methods, that urinary α2M* was strongly associated with total urinary protein (Spearman's Rho 0.76, p=0.03, n=8). To attenuate the influence of proteinuria on both α2M* and TGFβ1, a subset of 18 patients with median proteinuria during follow-up below 2 g/g of creatinine was analyzed. Patient clinical characteristics are shown in Table 1.

TABLE 1
Patient characteristics

| | |
|---|---|
| Number | 18 |
| Male, n (%) | 16 (89) |
| Age (years) | 70 ± 8 |
| Baseline eGFR (mL/min/1.73 m²) | 25 ± 10 |
| eGFR decline rate (mL/min/1.73 m²/year) | −2 ± 2 |
| Follow-up period (year) | 2.3 ± 0.6 |
| Protein/creatinine ratio (g/g) | 1.3 ± 0.5 |
| SBP (mmHg) | 142 ± 14 |
| DBP (mmHg) | 66 ± 8 |
| RASB use, n (%) | 17 (94) |

SBP, systolic blood pressure; DBP, diastolic blood pressure; RASB, renin-angiotensin system blocker.
Normally distributed values are presented as mean ± standard deviation.

α2M* was determined in 56 samples. As shown in FIG. 7B, the median value of urinary α2M* for each individual was associated with the median value of urinary TGFβ1. The association between α2M* and proteinuria was not significant in this low-proteinuria subgroup (p=0.16, Pearson correlation).

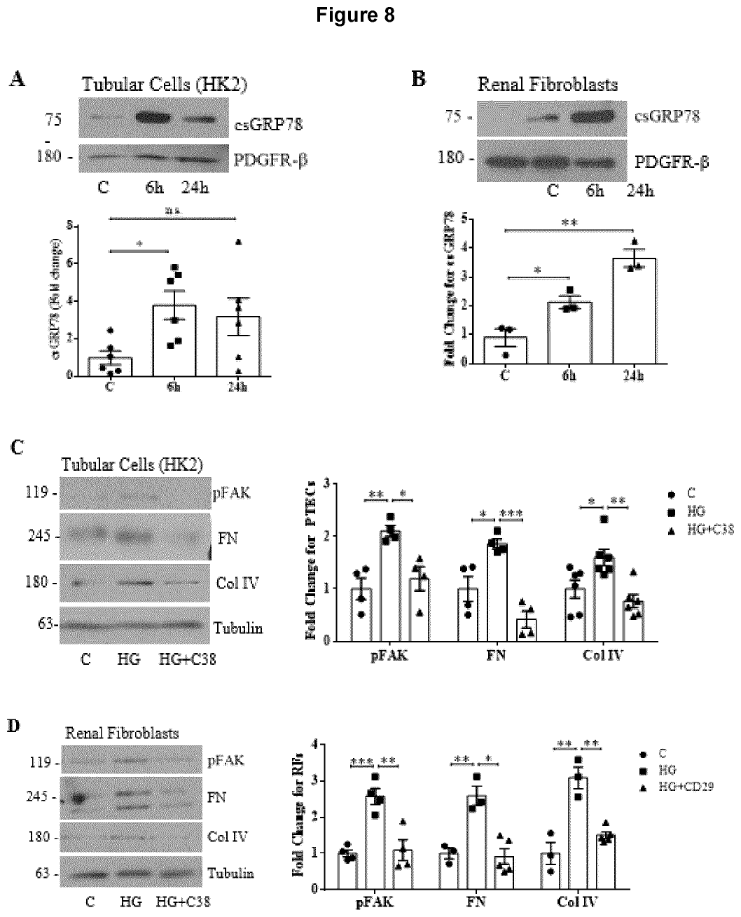

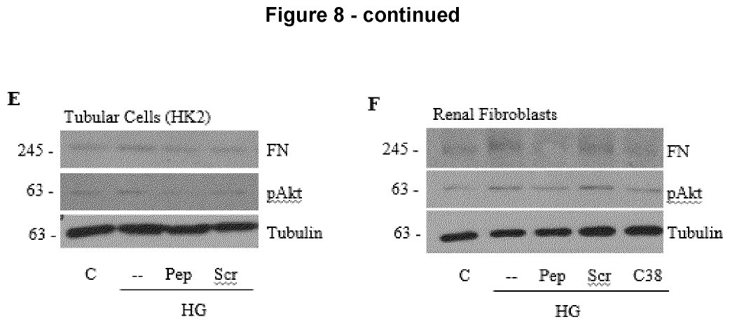

Cell surface GRP78/α2M* are important in cell types involved in tubulointerstitial fibrosis seen in later stage DKD—Fibrosis in later stages of DKD is seen in the tubular-interstitial area, involving signaling and profibrotic responses by both the tubular cells and renal fibroblasts. Since tubulointerstitial fibrosis correlates strongly with kidney disease progression to end-stage kidney disease³⁵, we determined whether α2M*/csGRP78 signaling can also regulate the profibrotic response in tubular cells and fibroblasts. We used a human proximal tubular cell line HK2 and rat renal fibroblasts. FIG. 8A-B shows that csGRP78 is strongly increased by HG in both cell types. We next used an antibody to the C-terminus of GRP78 termed C38 which was shown to prevent csGRP78 interaction with α2M* and downstream signaling in cancer cells³⁶. This prevented HG-induced profibrotic signaling (activation of FAK, upstream of Akt activation⁷, as well as matrix upregulation in both cell types (FIG. 8C-D). Importantly, the inhibitory peptide also prevented signaling (Akt activation) and matrix upregulation in fibroblasts, proximal tubular cells and renal fibroblasts, cells primarily responsible for interstitial matrix deposition (FIG. 8E/F).

To investigate the in vivo relevance of these findings, we determined whether α2M* was increased in the tubulointerstitium of diabetic kidneys. Images from the type 1 and type 2 DKD models respectively, and the human DKD biopsy sample were analyzed. Images were focused on the tubulointerstitial area and demonstrate clear increase in α2M* with diabetes, which was progressively increased with duration of diabetes in the type 1 Akita model. Increased α2M* was also seen in this region in kidneys of db/db mice, a model of type 2 DKD. Using immunofluorescence in unpermeabilized kidney tissue sections, we next showed a significant increase in csGRP78 in the tubules of 40-week type 1 diabetic Akita mice. Cell surface localization was confirmed by colocalization with the plasma membrane marker WGA, as seen in the mask which showed regions of overlay.

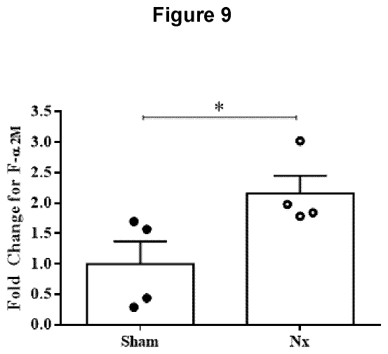

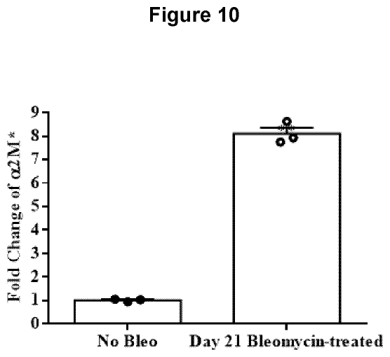

α2M is increased in CKD, lung and liver fibrosis—It was sought to investigate whether α2M* expression was increased in other models of fibrosis. First we assessed the non-diabetic CKD model in which mice undergo resection of ⅚ of their renal mass (⅚ nephrectomy, Nx). Compared to sham operated mice, we observed a significant increase in α2M* expression in the ⅚ Nx model (FIG. 9). Next we evaluated a bleomycin-induced mouse model of idiopathic lung fibrosis (IPF). In FIG. 10 significantly increased expression of α2M* was found only in bleomycin-treated mice at 21 days. We further evaluated a role for α2M in IPF through analysis of a scRNA-seq dataset of IPF compared to control patients. α2M expression is increased in IPF patients. This increase is localized to macrophages and myofibroblasts, cells which are critical to the fibrotic process. Lastly, we examined α2M expression in a publicly available scRNA-seq dataset from patients with human non-alcoholic steatohepatitis (NASH)/liver fibrosis, compared with control patients. We observed a significant increase in α2M expression in patients with NASH, localized to cell populations that contribute to fibrosis: mesoethelial cells (fibroblast precursors), scar-associated macrophages and dendritic cells.

Together, these data identify an important role for α2M* in mediating glucose-induced TGFβ1 upregulation in vitro, and suggest the clinical relevance of this finding. Furthermore, data in other renal and non-renal fibrotic models support a broader role for α2M* in mediating fibrosis and its inhibition as a potential anti-fibrotic therapeutic target.

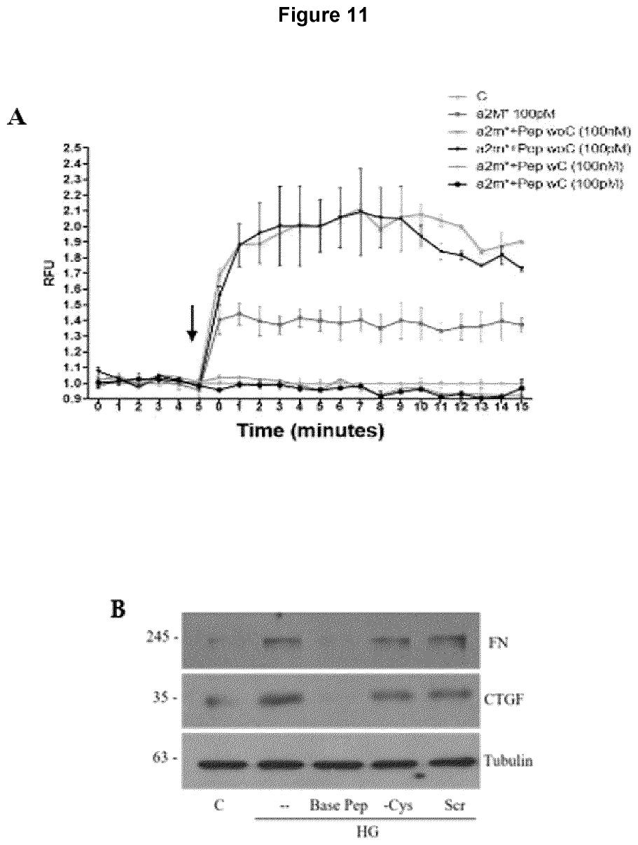

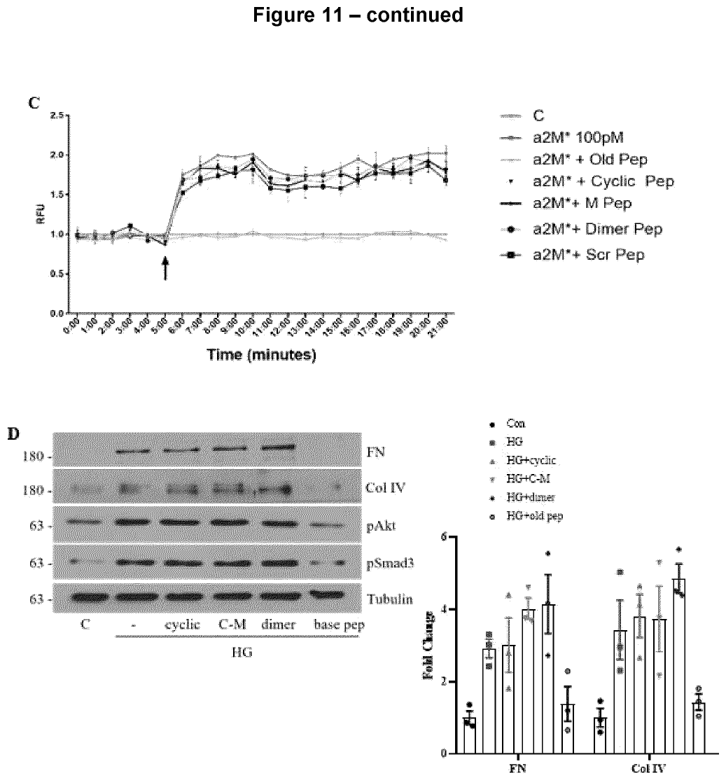

The N-terminal cysteine is critical to peptide inhibitory function—The importance of the N-terminal cysteine in the inhibitory peptide was previously unknown. To test this, a peptide lacking this amino acid was generated and tested for its inhibitory activity. FIG. 11A-B show that this peptide did not inhibit calcium signaling in 1LN cells induced by α2M* (arrow indicates its addition) nor did it prevent HG-induced profibrotic responses in MC. In the calcium assay (FIG. 11A), two concentrations of peptide were tested, 100 pM and 100 nM. To test whether methionine could replace the N-terminal cysteine given that it provides some redox function, we next tested a peptide in which the cysteine was replaced by a methionine. FIG. 11C-D shows that methionine cannot replace cysteine functionally in either of the two assays used. Two modifications to potentially increase in vivo stability were also tested, including dimerization through the cysteine and head to tail cyclization via an amide which leaves the cysteine intact. Neither of these peptides were functional (FIG. 11C-D). Taken together, the N-terminal cysteine and tertiary structure are critical to functionality of the peptide in inhibiting α2M* signaling via csGRP78 and the HG-induced profibrotic responses.

### DISCUSSION

It was recently demonstrated that csGRP78 is increased in diabetic kidneys and showed its importance in mediating HG-induced profibrotic signaling in MC (Van Krieken et al. Cell surface expression of 78-kDa glucose-regulated protein (GRP78) mediates diabetic nephropathy. Published online 2019. doi: 10.1074/jbc.RA118.006939). However, the mechanism by which csGRP78 is activated in this setting was unknown. We now identify α2M*, a known ligand for csGRP78, as a critical mediator of this signaling in MC. Not only is α2M expression increased by HG in MC and in diabetic mouse and human kidneys, but more importantly, its activation enables its function as a signaling ligand to csGRP78. α2M knockdown or α2M* neutralization inhibits profibrotic Akt activation and downstream matrix and profibrotic cytokine production. These data support an important role for α2M* in the pathogenesis of diabetic glomerulosclerosis, an early hallmark of DKD.

Biotinylation results showed that α2M* itself increased csGRP78. This indicates that locally increased α2M* in response to HG may facilitate the presentation of GRP78 on the cell surface, in addition to its role as a ligand, and that α2M* may participate in a positive feedback loop, leading to an augmentation of csGRP78 signaling and thus potentiation of profibrotic signaling. Interestingly, it was observed that forced cell surface GRP78 expression was sufficient to increase Akt activation and upregulation of ECM proteins/CTGF and was augmented by HG. This was blocked by α2M* inhibition, supporting the involvement of α2M* in both basal and HG-induced signaling through csGRP78.

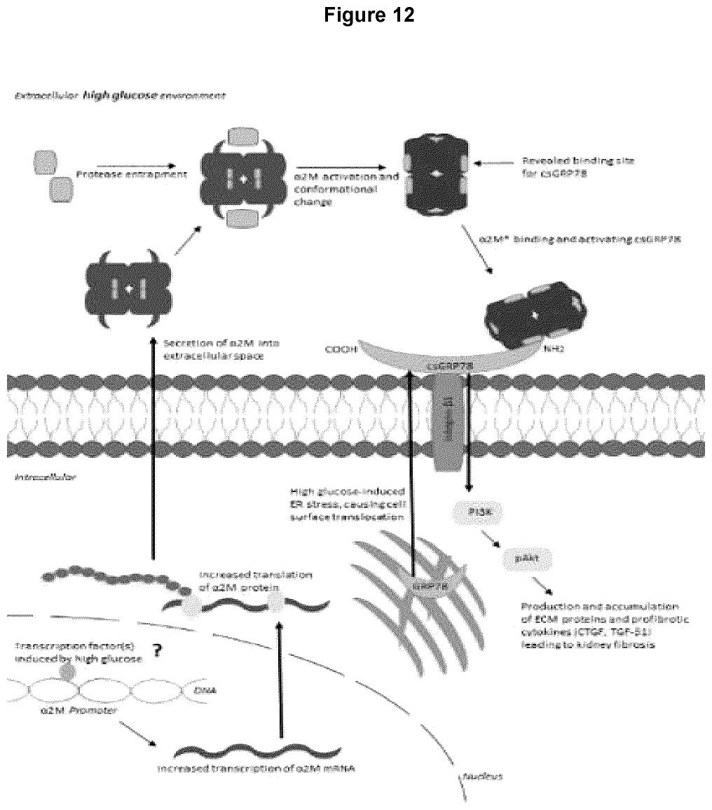

Taken together, it is herein shown that α2M/α2M* are increased in MC by HG and in diabetic glomeruli, and that α2M* regulates MC profibrotic responses through its interaction with csGRP78 (FIG. 12). These data indicated that blocking the α2M*/csGRP78 interaction is a novel therapeutic option for fibrotic conditions such as DKD. The data also support the therapeutic use of a peptide that specifically blocks α2M*/csGRP78 interaction. This peptide has unique characteristics, including the absolute requirement for an N-terminal cysteine, to impart function.

Evidence is also provided that α2M* is a valuable target for other fibrotic diseases including, but not limited to, non-diabetic CKD, IPF or NASH/liver fibrosis. The observed significant increase in α2M and/or α2M* expression in these fibrotic disorders supports that α2M* is a pathogenic regulator generally in fibrotic disease/conditions.

Different embodiments of the invention have been shown by the above example. Those skilled in the art could develop alternatives to the methods mentioned above that are within the scope of the invention and defined claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### REFERENCES

1. Reidy K. Kang H M, Hostetter T, Susztak K. Molecular mechanisms of Diabetic kidney disease. J Clin Invest. 2014; 124 (6): 2333-2340. doi: 10.1172/JCI72271
2. Diabetic Nephropathy Treatment & Management: Approach Considerations. Glycemic Control. Management of Hypertension. Accessed Apr. 9, 2020. https://emedicine.medscape.com/article/238946-treatment
3. Gross J L, de Azevedo M J, Silveiro S P, Canani L H, Caramori M L, Zelmanovitz T. Diabetic nephropathy: diagnosis, prevention, and treatment. Diabetes Care. 2005; 28 (1): 164-176. Accessed Dec. 13, 2018. http://www.ncbi.nlm.nih.gov/pubmed/15616252
4. Alicic R Z, Rooney M T, Tuttle K R, Diabetic Kidney Disease. Clin J Am Soc Nephrol. 2017; 12 (12): 2032-2045. doi: 10.2215/CJN. 11491116
5 Schlondorff D. Banas B. The Mesangial Cell Revisited: No Cell Is an Island. J Am Soc Nephrol. 2009; 20 (6): 1179-1187. doi: 10.1681/ASN.2008050549
6. Zhu G. Lee A S, Role of the unfolded protein response. GRP78 and GRP94 in organ homeostasis. J Cell Physiol. 2015; 230 (7): 1413-1420. doi: 10.1002/jcp.24923
7. Van Krieken R. Mehta N. Wang T. et al. Cell surface expression of 78-kDa glucose-regulated protein (GRP78) mediates diabetic nephropathy. Published online 2019. doi: 10.1074/jbc.RA118.006939
8. Wu S M, Pizzo S V, Hemostasis and Thrombosis. Basic Principles and Clinical Practice. Third Edit. (Colman R W, Hirsh J. Marder V J, Salzman E W, eds.). J. B. Lippencott. Williams and Williams: 2000. doi: 10.1373/49.2.345/-a
9. Rehman A A, Ahsan H. Khan F H, Alpha-2-macroglobulin: A physiological guardian. J Cell Physiol. 2013; 228 (8): 1665-1675. doi: 10.1002/jcp.24266
10. Cater J H, Wilson M R, Wyatt A R. Alpha-2-Macroglobulin, a Hypochlorite-Regulated Chaperone and Immune System Modulator. Oxid Med Cell Longev. 2019; 2019. doi: 10.1155/2019/5410657
11. Gopal U. Gonzalez-Gronow M. Pizzo S V. Activated α2-macroglobulin regulates transcriptional activation of c-MYC target genes through cell surface GRP78 protein. J Biol Chem. 2016; 291 (20): 10904-10915. doi: 10.1074/jbc.M115.708131
12. Gonzalez-Gronow M, Cuchacovich M, Llanos C, Urzua C, Gawdi G, Pizzo S V. Prostate Cancer Cell Proliferation In vitro Is Modulated by Antibodies against Glucose-Regulated Protein 78 Isolated from Patient Serum. Cancer Res. 2006; 66 (23): 11424-11455. doi: 10.1158/0008-5472.CAN-06-1721
13. Hart J P, Pizzo S V., Johnson C E, Misra U K, Gonzalez-Gronow M. Gawdi G. The Role of Grp 78 in a 2-Macroglobulin-induced Signal Transduction. J Biol Chem. 2002; 277 (44): 42082-42087. doi: 10.1074/jbc.m206174200
14. Kant Misra U. Gonzalez-Gronow M. Gawdi G. Wang F. Vincent Pizzo S. A novel receptor function for the heat shock protein Grp78: silencing of Grp78 gene expression attenuates α2M *-induced signallin[1] U. Kant Misra. M. Gonzalez-Gronow. G. Gawdi. F. Wang. and S. Vincent Pizzo. “A novel receptor function for the heat shock p. Cell Signal. 2004; 16 (8): 929-938. doi: 10.1016/j.cellsig.2004.01.003
15. Ni M. Zhang Y. Lee A S, Beyond the endoplasmic reticulum: atypical GRP78 in cell viability, signalling and therapeutic targeting. Biochem J. 2011; 434 (2): 181-188. doi: 10.1042/BJ20101569
16. Misra U K, Payne S, Pizzo S V. The Monomeric Receptor Binding Domain of Tetrameric a2-Macroglobulin Binds to Cell Surface GRP78 Triggering Equivalent Activation of Signaling Cascades. Biochemistry. 2013; 52 (23): 4014-4025. doi: 10.1021/bi400376s
17. Yang A H, Chen J Y. Glomerular deposition of alpha 2-macroglobulin in glomerular diseases. Nephrol Dial Transplant. 1997: 12 (3): 465-469. http://www.ncbi.nlm.nih.gov/pubmed/9075125
18. James K, Merriman J, Gray R S, Duncan L J, Herd R. Serum alpha 2-macroglobulin levels in diabetes. J Clin Pathol. 1980; (August 1979): 163-166.
19. Rojas-Alcayaga G, Aitken J P, Baeza M, et al. α-2-Macroglobulin in Saliva Is Associated with Glycemic Control in Patients with Type 2 Diabetes Mellitus. Dis Markers. 2015; 2015:1-5. doi: 10.1155/2015/128653
20. Menon R, Otto E A, Hoover P J, et al. Single cell transcriptomics identifies focal segmental glomerulosclerosis remission endothelial biomarker. JCI insight. 2020; 5 (6). doi: 10.1172/jci.insight. 133267
21. Ramachandran P. Dobie R. Wilson-Kanamori J. et al. Resolving the fibrotic niche of human liver cirrhosis at single cell level. Nature. 2019: 575 (7783): 512. doi: 10.1038/S41586-019-1631-3
22. Krepinsky J C, Ingram A J, Tang D, Wu D, Liu L, Scholey J W. Nitric Oxide Inhibits Stretch-Induced MAPK Activation in Mesangial Cells Through RhoA Inactivation. Published online 2003. doi: 10.1097/01.ASN.0000094085.04161.A7
23. Biltoft D, Gram J B, Larsen A, et al. Fast form alpha-2-macroglobulin-A marker for protease activation in plasma exposed to artificial surfaces. Clin Biochem. 2017; 50 (18): 1203-1208. doi: 10.1016/j.clinbiochem.2017.09.002
24. Van Krieken R. Marway M. Parthasarathy P. et al. Inhibition of SREBP With Fatostatin Does Not Attenuate Early Diabetic Nephropathy in Male Mice. Endocrinology. 2018: 159 (3): 1479-1495. doi: 10.1210/en.2018-00093
25. Zhang Y, Liu R, Ni M, Gill P, Lee A S. Cell surface relocalization of the endoplasmic reticulum chaperone and unfolded protein response regulator GRP78/BiP. J Biol Chem. 2010; 285 (20): 15065-15075. doi: 10.1074/jbc.M109.087445
26. Verhave J C, Bouchard J, Goupil R, et al. Clinical value of inflammatory urinary biomarkers in overt diabetic nephropathy: A prospective study. Diabetes Res Clin Pract. 2013; 101 (3): 333-340. doi: 10.1016/j.diabres.2013.07.006
27. Hartner A. Schöckliviann H. Pröls F. Müller U. Bernd Sterzel R. α8 integrin in glomerular mesangial cells and in experimental glomerulonephritis. In; Kidney International. Vol 56. Blackwell Publishing Inc.: 1999; 1468-1480. doi: 10.1046/j. 1523-1755.1999.00662.x
28. Sheu M L·Shen C C·Jheng J R·Chiang C K·Activation of PI3K in response to high glucose leads to regulation of SOCS-3 and STAT⅓ signals and induction of glomerular mesangial extracellular matrix formation. Oncotarget. 2017; 8 (10): 16925-16938. doi: 10.18632/oncotarget. 14808
29. Toda N. Mukoyama M. Yanagita M. Yokoi H. CTGF in kidney fibrosis and glomerulonephritis. Inflamm Regen. 2018: 38 (1). doi: 10.1186/s41232-018-0070-0
30. Gopal U, Pizzo S V, Cell surface GRP78 promotes tumor cell histone acetylation through metabolic reprogramming: a mechanism which modulates the Warburg effec1. Gopal U, Pizzo S V, Cell surface GRP78 promotes tumor cell histone acetylation through metabolic reprogramming: a m. Oncotarget. 2017; 8 (64): 107947-107963. doi: 10.18632/oncotarget.22431
31. Zhao S, Li H, Wang Q, et al. The role of c-Src in the invasion and metastasis of hepatocellular carcinoma cells induced by association of cell surface GRP78 with activated α2M. BMC Cancer. 2015; 15 (1): 1-14. doi: 10.1186/s12885-015-1401-2
32. Chang A S, Hathaway C K, Smithies O, Kakoki M. Transforming growth factor-β1 and diabetic nephropathy. Am J Physiol-Ren Physiol. 2016; 310 (8): F689-F696. doi: 10.1152/ajprenal.00502.2015
33. Li J H, Huang X R, Zhu H J, Johnson R, Lan H Y. Role of TGF-β signaling in extracellular matrix production under high glucose conditions. Kidney Int. 2003; 63 (6): 2010-2019. doi: 10.1046/j. 1523-1755.2003.00016.x
34. Wu D, Peng F, Zhang B, Ingram A J, Gao B, Krepinsky J C. Collagen I induction by high glucose levels is mediated by epidermal growth factor receptor and phosphoinositide 3-kinase/Akt signalling in mesangial cells. Diabetologia. 2007; 50 (9): 2008-2018. doi: 10.1007/s00125-007-0721-1
35. Humphreys B D, Mechanisms of Renal Fibrosis. 2018; 80:309-326. https: doi.org 101146 annurev-physiol-022516-034227. doi: 10.1146/ANNUREV-PHYSIOL-022516-034227
36. De Ridder G G, Ray R, Pizzo S V. A murine monoclonal antibody directed against the carboxyl-terminal domain of GRP78 suppresses melanoma growth in mice. Melanoma Res. 2012; 22 (3): 225-235. doi: 10.1097/CMR. 0b013e32835312fd
37. Yoshino S, Fujimoto K, Takada T, et al. Molecular form and concentration of serum a2-macroglobulin in diabetes. Sci Rep. 2019; 9 (1): 1-10. doi: 10.1038/s41598-019-49144-7
38. Ahmad J, Singh M, Saleemuddin M, A Study of Plasma Alpha-2-Macroglobulin Levels in Type 2 Diabetic Subjects with Microalbuminuria. J Assoc Physicians India. 2001; 49 (NOV): 1062-1065. Accessed Dec. 28, 2020. https://europepmc.org/article/med/11868856
39. French K, Yerbury J J, Wilson M R. Protease activation of a2-macroglobulin modulates a chaperone-like action with broad specificity. Biochemistry. 2008; 47 (4): 1176-1185. doi: 10.1021/bi701976f
40. Kim K M, Chung K W, Jeong H O, et al. MMP2-A2M interaction increases ECM accumulation in aged rat kidney and its modulation by calorie restriction. Oncotarget. 2018; 9 (5): 5588-5599. doi: 10.18632/oncotarget.23652
41. van Goor H, Diamond J R, Ding G, Kaysen G. Alpha Macroglobulins and the Low-Density-Lipoprotein-Related Protein/Alpha-2-Macroglobulin Receptor in Experimental Renal Fibrosis. Nephron Exp Nephrol. 1999; 7 (1): 35-43. doi: 10.1159/000020582
42. Rodríguez-Calvo R, Ferrán B, Alonso J, et al. NR4A receptors up-regulate the antiproteinase alpha-2 macroglobulin (A2m) and modulate MMP-2 and MMP-9) in vascular smooth muscle cells. Thromb Haemost. 2015; 113 (6): 1323-1334. doi: 10.1160/TH14-07-0645
43. Lin L, Hu K. LRP-1: Functions, signaling and implications in kidney and other diseases. Int J Mol Sci. 2014; 15 (12): 22887-22901. doi: 10.3390/ijms151222887
44. Yoo J, Wang W, Desiderio S, Nathans D. Synergistic Activity of STAT3 and c-Jun at a Specific Array of DNA Elements in the [symbol] 2-Macroglobulin Promoter * 2001; 276 (28): 26421-26429. doi: 10.1074/jbc.M009935200
45. Bao L, Devi S, Bowen-Shauver J, Ferguson-Gottschall S, Robb L, Gibori G. The role of interleukin-11 in pregnancy involves up-regulation of α2-macroglobulin gene through Janus kinase 2-signal transducer and activator of transcription 3 pathway in the decidua. Mol Endocrinol. 2006; 20 (12): 3240-3250. doi: 10.1210/me.2006-0296
46. Brosius F C, Tuttle K R, Kretzler M. JAK inhibition in the treatment of diabetic kidney disease. Diabetologia. 2016; 59 (8): 1624-1627. doi: 10.1007/s00125-016-4021-5
47. Weigert C, Sauer U, Brodbeck K, Pfeiffer A, H H U, Schleicher E D. AP-1 Proteins Mediate Hyperglycemia-Induced Activation of the Human TGF-1 Promoter in Mesangial Cells.; 2000.
48. Misra U K, Payne S, Pizzo S V. Ligation of Prostate Cancer Cell Surface GRP78 Activates a Proproliferative and Antiapoptotic Feedback Loop. J Biol Chem. 2010; 286 (2): 1248-1259. doi: 10.1074/jbc.m110.129767
49. Hinnen D. Glucagon-like peptide 1 receptor agonists for type 2 diabetes. Diabetes Spectr. 2017; 30 (3): 202-210. doi: 10.2337/ds16-0026

1. A method of treating or inhibiting the development of fibrosis in a mammalian subject, comprising administering to the subject a therapeutically effective amount of an entity that disrupts the interaction between activated alpha 2-macroglobulin (α2M*) and cell surface 78 kDa glucose regulated protein csGRP78.

2. The method of claim 1, wherein the entity is selected from a polynucleotide, protein or peptide.

3. The method of claim 1, wherein the entity is a polynucleotide that inhibits upregulation of alpha 2-macroglobulin (α2M) which is associated with fibrosis.

4. The method of claim 1, wherein the entity is an antibody that binds to α2M, α2M*or csGRP78.

5. The method of claim 1, wherein the entity is a α2M*/csGRP78 antifibrotic peptide derived from the α2M* binding site in csGRP78.

6. The method of claim 5, wherein the peptide is derived from the csGRP78 peptide, LIGRTWNDPSVQQDIKFL, and i) comprises an N-terminal cysteine residue, ii) comprises at least 10 consecutive amino acid residues of the csGRP78 peptide, and iii) retains the tertiary structure of the csGRP78 peptide.

7. The method of claim 6, wherein the antifibrotic peptide exhibits at least 75% sequence identity with the csGRP78 peptide.

8. The method of claim 1, wherein the fibrosis is fibrosis of the lung, kidney, heart, skin or bone marrow.

9. The method of claim 1, wherein the subject is a human.

10. The method of claim 1, wherein the subject has diabetic or non-diabetic kidney disease, idiopathic pulmonary fibrosis or NASH/liver fibrosis.

11. An anti-fibrotic peptide derived from the csGRP78 peptide, LIGRTWNDPSVQQDIKFL, wherein the anti-fibrotic peptide i) comprises an N-terminal cysteine residue, ii) comprises at least 10 consecutive amino acid residues of the csGRP78 peptide, iii) retains the tertiary structure of the csGRP78 peptide, and iv) comprises at least one amino acid substitution, deletion or addition to the csGRP78 peptide.

12. The anti-fibrotic peptide of claim 11, wherein one or more amino acids are modified to incorporate a reactive group that functions to form a covalent bond with α2M*.

13. The anti-fibrotic peptide of claim 12, wherein the reactive group is selected from acrylamides, ester derivatives, α,β-unsaturated carbonyl compounds, α,β-unsaturated amides and sulfonyl fluoride derivatives.

14. The anti-fibrotic peptide of claim 11, which is phosphorylated, glycosylated or modified by lipidation.

15. The anti-fibrotic peptide of claim 11, comprising an N-terminal and/or C-terminal protecting group.

16. An anti-fibrotic peptide derived from the csGRP78 peptide, LIGRTWNDPSVQQDIKFL, wherein the anti-fibrotic peptide i) comprises an N-terminal cysteine residue, ii) comprises at least 10 consecutive amino acid residues of the csGRP78 peptide, iii) retains the tertiary structure of the csGRP78 peptide, and iv) is conjugated to one or more entities that form a covalent bond with α2M* or that target fibrotic tissue, or is packaged in a delivery system that targets fibrotic tissue.

17. The anti-fibrotic peptide of claim 16, wherein the entity is a reactive group selected from the group consisting of acrylamides, ester derivatives, α,β-unsaturated carbonyl compounds, α,β-unsaturated amides and sulfonyl fluoride derivatives.

18. The anti-fibrotic peptide of claim 16, wherein the entity is an antibody or fragment thereof that targets a protein expressed by fibrotic tissue.

19. The anti-fibrotic peptide of claim 16, wherein the entity is a ligand that targets cells of a fibrotic organ.

20. The anti-fibrotic peptide of claim 19, wherein the ligand is an antibody or fragment thereof that targets the fibronectin expressed in fibrotic kidney.

21. The anti-fibrotic peptide of claim 16, wherein the entity is a ligand that binds serum pan IgG antibodies.

22. A composition comprising the anti-fibrotic peptide of any one of claims 11-21 in combination with a pharmaceutically acceptable carrier.

23. A composition comprising:
1) an anti-fibrotic peptide derived from the csGRP78 peptide, LIGRTWNDPSVQQDIKFL, wherein the anti-fibrotic peptide i) comprises an N-terminal cysteine residue, ii) comprises at least 10 consecutive amino acid residues of the csGRP78 peptide, iii) retains the tertiary structure of the csGRP78 peptide, and
2) a second therapeutic agent.

24. The composition of claim 23, wherein the second therapeutic agent is anti-fibrotic agent.

25. The composition of claim 23, wherein the second therapeutic agent is an agent useful to treat diabetes.

26. The composition of claim 25, wherein the second therapeutic agent is selected from the group of an incretin hormone, metformin, an SGLT2 inhibitor, sulfonylurea, or N-acetyl-seryl-aspartyl-lysyl-proline.
